(19) United States
(12) Patent Application Publication
(10) Pub. No.: US 2026/0198801 A1
(43) Pub. Date: Jul. 16, 2026

(54) MEASUREMENT DEVICE AND METHOD THEREOF

(71) Applicant: FEMTHERAPEUTICS, Montreal (CA)

(72) Inventors: Negin ASHOURI, Montreal (CA); Mihnea GANGAL, Montreal (CA); Alireza SABOUKHI, Montreal (CA); Majid ROSHANFAR, Montreal (CA); Erfan FATEHI, Montreal (CA); Inara LALANI, Montreal (CA); Lydia Yolanda AGUIRRE PERALES, Montreal (CA); Vincent CASTONGUAY-SUI, Montreal (CA)

(21) Appl. No.: 19/134,400

(22) PCT Filed: Dec. 1, 2023
(86) PCT No.: PCT/CA2023/051607

§ 371 Date: May 30, 2025

Related U.S. Application Data
(60) Provisional application No. 63/429,722, filed on Dec. 2, 2022.
(60) Provisional application No. 63/504,411, filed on May 25, 2023.
(60) Provisional application No. 63/516,896, filed on Aug. 1, 2023.

(51) Int. Cl. A61B 5/107; A61B 5/00

(52) U.S. Cl. CPC A61B 5/1072; A61B 5/1076; A61B 5/4331; A61B 5/4337; A61B 5/6824; A61B 5/6825; A61B 2560/0223

(57) ABSTRACT

The present application discloses a measurement device including a first finger connector for connecting to a first finger; a second finger connector for connecting to a second finger; a string extending between the first finger connector and the second finger connector, and having an extension portion extending beyond at least one of the first and second finger connectors by an extension length; wherein a separation distance of the first and second finger connectors is proportional to the extension length. A kit and a method of using said measurement device is further disclosed.

### TECHNICAL FIELD

The present technology generally relates to a measurement device for obtaining measurements in an anatomical space of a patient, such as but not limited to obtaining gynecological measurements during a pelvic examination.

### BACKGROUND

Physicians performing pelvic examinations obtain a variety of measurements and other data to provide personalized healthcare for a patient.

One instance is for selecting gynecological prosthetics, such as a pessary or other intravaginal devices, or for designing a personalized gynecological prosthetic, according to the data obtained about the vaginal canal. During pelvic examinations, physicians obtain multiple measurements of the vaginal canal to aid in the fitting and development of the pessary and/or pelvic prosthetics. Currently, physicians insert their fingers into the patient's vaginal canal and measure distances by separating their fingers while contacting anatomical landmarks. The physician withdraws their fingers and chooses a pessary size which best approximates the distances. As such, selection of the pessary size is subjective and inconsistent, leading to complications with long-term use such as irritation, lacerations, sores, displacement, discharge, chronic pain, and ultimately a failed prosthetic fitting resulting in discontinued use.

In another instance, during labor and delivery, physicians monitor and measure cervical dilation. Cervical dilation measurements are not standardized, and different physicians use different methods to measure dilation. Some physicians will assess dilation by tactile feedback, inserting two fingers into the vagina and will estimate dilation by feeling the cervix, resulting in subjective measurements which vary between physicians and are inaccurate. This can lead to medical decisions being taken which are not appropriate to the patient and can endanger the life of the patient or the baby. In some instances, the same patient may be seen by different physicians who use different techniques to assess cervical dilation, and this can result in complications during labour.

Alternatively, tools have been developed such as mechanical calipers or three-dimensional ultrasonograms. However, these tools are complex, cause discomfort to the patient, are difficult to introduce into clinical practice, and may be inaccurate (for example by distorting the cervix to obtain the measurements) or not reproducible.

In both instances, whether using tactile feedback or tools, inaccurate and inconsistent measurements could result in the physician inducing labor at too early a stage leading to various complications including: increased length of labor, cesarean sections, post partum hemorrhage, risks related to blood transfusion, infection, uterine rupture, fetal distress, etc. The physician may also introduce unnecessary drugs based on inaccurate measurements.

Thus, there is a desire for a measurement device which overcomes at least some of the abovementioned problems.

### SUMMARY

It is an object of the present technology to ameliorate at least some of the inconveniences present in the prior art.

According to one aspect of the present technology, aspects and embodiments of the present technology are defined in the appended claims.

In one broad aspect of the present technology, a measurement device is provided. The measurement device includes a first finger connector for connecting to a first finger; a second finger connector for connecting to a second finger; a string extending between the first finger connector and the second finger connector, and having an extension portion extending beyond at least one of the first and second finger connectors by an extension length; wherein a separation distance of the first and second finger connectors is proportional to the extension length.

In some embodiments, the string is anchored with respect to one of the first and second finger connectors and is slidingly moveable with respect to the other of the first and second finger connectors.

In some embodiments, the measurement device further includes an indicator for indicating a measure of the separation distance or the extension length.

In some embodiments, the extension portion is connected to a spool assembly, the extension length being proportional to a spooled length, the indicator having one or more of: visual markings on the string or the spool assembly, tactile markings on the string or the spool assembly, and a digital/mechanical output of the spooled length.

In some embodiments, the spool assembly includes a tape measure device which is wearable on a wrist of the user.

In some embodiments, the visual markings include one or more of: colours, indicia, letters or numbers.

In some embodiments, the indicator includes one or more of: visual markings on the string and tactile markings on the string.

In some embodiments, the visual markings and/or the tactile markings are on the string.

In some embodiments, the measurement device further includes a hand covering, wherein the first finger connector and the second finger connector are integral with or disposed in relation to the hand covering.

In some embodiments, the hand covering is disposed over the first and the second finger connectors, the hand covering having a first hand covering finger housing the first finger connector, the first hand covering finger having a first hand covering opening; a second hand covering finger housing the second finger connector, the second hand covering finger having a second hand covering opening; and the string extending through the first hand covering opening and the second hand covering opening.

In some embodiments, the hand covering is disposed beneath the first and the second finger connectors, the hand covering comprising: a first hand covering finger housed within the first finger connector; and a second hand covering finger housed within the second finger connector.

In some embodiments, the measurement device further includes an additional hand covering, wherein the additional hand covering is disposed beneath the first and the second finger connectors.

In some embodiments, the hand covering further includes measurement markings disposed along an exterior surface of the hand covering, which markings may extend along one or more of the hand covering fingers and/or along a wrist portion of the hand covering.

In some embodiments, the hand covering is reversible such that the hand covering can be worn on either a left or a right hand.

In some embodiments, the measurement device further comprises a guiding member configured to receive the extension portion, the guiding member comprising a first end portion, a second end portion, and an intermediate portion therebetween.

In some embodiments, the guiding member comprises a tube having a channel extending therethrough configured to receive the extension portion.

In some embodiments, when the first finger connector is connected to the first finger of the user, and the second finger connector is connected to the second finger of the user: the first end portion is disposed along the first finger; and the second end portion is disposed along a wrist portion of the hand covering.

In some embodiments, the intermediate portion is disposed along a third finger of the hand covering.

In some embodiments, the first finger connector is connected to the first finger, and the second finger connector is connected to the second finger: the first end portion is disposed along a portion of the first finger of the hand covering; the second end portion is disposed along a portion of a third finger of the hand covering.

In some embodiments, the third finger of the hand covering is configured to receive a thumb when the measurement device is in use.

In some embodiments, the first end portion arcs over a distal tip of the first fingertip when the measurement device is in use.

In some embodiments, the first end portion comprises an opening, and the string extends through the opening.

In some embodiments, the guiding member and/or the string is lubricated.

In some embodiments, the guiding member comprises an injection port for injecting lubricant.

In some embodiments, the first finger connector includes a first distal tip having a first center portion; the second finger connector includes a second distal tip having a second center portion; and the string extends from the first center portion of the first finger connector to the second center portion of the second finger connector.

In some embodiments, an end region of the string is anchored in relation to the second finger connector.

In some embodiments, the first finger connector includes a first finger cap configured to accommodate a first fingertip of the first finger, and the second finger connector includes a second finger cap configured to accommodate a second fingertip of the second finger.

In some embodiments, the first finger cap includes a first finger cap opening, and the string is disposed beneath the first finger cap and extends through the first finger cap opening.

In some embodiments, the second finger cap includes a second finger cap opening, and the string extends from the first finger cap through the second finger cap opening.

In some embodiments, the string is affixed on or beneath the second finger cap.

In some embodiments, the measurement device further comprises an interdigit guiding member disposed between the first finger connector and the second finger connector, the interdigit guiding member comprising at least one pair of retaining arms for releasably retaining the portion of the string extending between the first and second finger connectors such that the string extends along the interdigit guiding member.

In some embodiments, the pair of retaining arms extend an entire length of the interdigit guiding member.

In some embodiments, the interdigit guiding member includes a plurality of pairs of retaining arms spaced apart along the interdigit guiding member.

In some embodiments, the measurement device is configured to store the separation distance in a patient database or to cause the storing of the separation distance.

In some embodiments, the measurement device further includes a locking mechanism for securing a position of the extension portion of the string relative to one of the first and second finger connectors.

In some embodiments, the locking mechanism is disposed along the extension portion of the string.

In some embodiments, the locking mechanism presses down on a segment of the extension portion of the string upon actuation of the locking mechanism.

In some embodiments, the first finger is an index finger and the second finger is a middle finger.

In another broad aspect of the present technology, a measurement device is provided. The measurement device includes a hand covering having a first finger portion for a first finger; a second finger portion for a second finger; a string extending between the first finger portion and the second finger portion, and having an extension portion extending beyond at least one of the first and second finger portions by an extension length; wherein a separation distance of the first and second finger portions is proportional to the extension length.

In some embodiments, the string is anchored with respect to one of the first and second finger portions and is slidingly moveable with respect to the other of the first and second finger portions.

In some embodiments, the measurement device further includes an indicator for indicating a measure of the separation distance or the extension length.

In some embodiments, the extension portion is connected to a spool assembly, the extension length being proportional to a spooled length, the indicator having one or more of: visual markings on the string or the spool assembly, tactile markings on the string or the spool assembly, and a digital/mechanical output of the spooled length.

In some embodiments, the spool assembly includes a tape measure device which is wearable on a wrist of the user.

In some embodiments, the visual markings include one or more of: colours, indicia, letters or numbers.

In some embodiments, the indicator includes one or more of: visual markings on the string and tactile markings on the string.

In some embodiments, the visual markings and/or the tactile markings are on the string.

In some embodiments, the hand covering further includes measurement markings disposed along an exterior surface of the hand covering.

In some embodiments, the hand covering can be worn on either a left or a right hand.

In some embodiments, the measurement device further includes a guiding member configured to receive at least a portion of the extension portion, the guiding member having a first end portion, a second end portion, and an intermediate portion therebetween.

In some embodiments, the guiding member includes a tube having a channel extending therethrough in which the at least a portion of the extension portion is received.

In some embodiments, the first end portion is disposed along the first finger of the hand covering; and the second end portion is disposed along a wrist portion of the hand covering.

In some embodiments, the intermediate portion is disposed along a third finger of the hand covering.

In some embodiments, the first end portion is disposed along the first finger of the hand covering; and the second end portion is disposed along a third finger of the hand covering.

In some embodiments, the first end portion arcs over a distal tip of the first fingertip of the user when the hand covering is worn on a hand of the user.

In some embodiments, there is an opening defined in the first end portion through which the string extends.

In some embodiments, the guiding member and/or the string is lubricated.

In some embodiments, the guiding member comprises an injection port for injecting lubricant.

In some embodiments, the first finger portion includes a first distal tip having a first center portion; the second finger portion includes a second distal tip having a second center portion; and the string extends from the first center portion of the first finger portion to the second center portion of the second finger portion.

In some embodiments, an end region of the string is anchored in relation to the second finger portion.

In some embodiments, the first finger portion comprises a first finger cap opening, and the string extends through the first finger cap opening.

In some embodiments, the string is connected to the second finger cap.

In some embodiments, the measurement device further includes an interdigit guiding member disposed between the first finger portion and the second finger portion, the interdigit guiding member having at least one pair of retaining arms for releasably retaining the portion of the string extending between the first and second finger portions such that the string extends along the interdigit guiding member.

In some embodiments, the pair of retaining arms extend an entire length of the interdigit guiding member.

In some embodiments, the interdigit guiding member includes a plurality of pairs of retaining arms spaced apart along the interdigit guiding member.

In some embodiments, the measurement device is configured to store the separation distance or to cause the storing of the separation distance.

In some embodiments, the measurement device further includes a locking mechanism for securing a position of the extension portion of the string relative to one of the first and second finger portions.

In some embodiments, the locking mechanism is disposed along the extension portion of the string.

In some embodiments, the locking mechanism presses down on a segment of the extension portion of the string upon actuation of the locking mechanism.

In some embodiments, when the hand covering is worn on the hand of the user, the first finger portion is configured to be disposed relative to an index finger of the user and the second finger is configured to be disposed relative to a middle finger of the user.

In another broad aspect of the present technology, a kit is provided. The kit includes the measurement device as previously described; and a calibration tool for calibrating the measurement device.

In another broad aspect of the present technology, a kit is provided. The kit includes a plurality of the measurement devices as previously described, each of the plurality of measurement device being different sizes, accommodating for different hand sizes; and each of the plurality of measurement devices being pre-calibrated.

In another broad aspect of the present technology, a method of using a measurement device is provided. The method includes inserting at least a portion of a measurement device into a patient, the measurement device having first and second finger connectors, a string extending between the first and second finger connectors and having an extension portion extending beyond one of the first and second connectors, and an indicator associated with the extension portion; positioning the first finger connector at a first target location; positioning the second finger connector at a second target location so that the first and second connectors are separated by a separation distance; and determining the separation distance between the first finger connector and the second finger connector based on an extension length of the extension portion, which extension length can be determined from outside of the patient.

In some embodiments, positioning the first finger connector of the measuring device at the first target location includes positioning a first center portion of a first distal tip of the first finger connector at the first target location.

In some embodiments, positioning the second finger connector of the measuring device at the second target location includes positioning a second center portion of a second distal tip of the second finger connector at the second target location.

In some embodiments, the indicator includes a spooled tape measure connected to an end of the extension portion of the string and determining the extension length includes reading a spooled length from the tape measure, the spooled length being proportional to the separation distance.

In some embodiments, the indicator includes at least one of visual markings and tactile markings disposed on the extension portion; and determining the extension length includes reading at least one of the visual markings and the tactile markings from the extension portion.

In some embodiments, the method further includes calibrating the measurement device using a calibration tool.

In some embodiments, the method further includes releasing the string from retention arms of the measuring device.

In some embodiments, releasing the string from the retention arms includes pulling a first end of the string.

In some embodiments, the method further includes moving the second finger connector towards a third target location, such the extension portion of the string of the measurement device extends a second extension length; determining the second extension length; and determining a second separation distance between the first finger connector and the second finger connector based on the second extension length.

In another broad aspect of the present technology, a measurement device is provided. The measurement device includes a first finger connector for connecting to a first finger; a second finger connector for connecting to a second finger; a sensing system having a first component connected to the first finger connector and a second component connected to the second finger connector; where the first and the second components are configured to detect a distance between one another.

In some embodiments, the first component is a first sensor and the second components is a second sensor; and the first and second sensors are configured to provide a signal indicative of the distance.

In some embodiments, the measurement device further includes a processor communicatively connected to the first and second sensors for receiving the signal.

In some embodiments, the measurement device further includes a display communicatively connected to the processor for displaying the distance.

In some embodiments, the first sensor is disposed at a distal portion of the first connector and the second sensor is disposed at a distal portion of the second connector.

In some embodiments, the first and the second sensor are at least one of: a Hall effect sensor, a Bluetooth, an RFID, and an inertial measurement unit.

In some embodiments, the sensing system is communicatively connected to a storage device for storing the distance detected by the first and the second sensors.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

As used herein, the term “hand covering” may refer to a glove, configured to fully cover a user's hand, or a partial glove, configured to cover a portion of a user's hand.

Additional and/or alternative features, aspects, and advantages of embodiments of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a perspective view from a top side of a measurement device when worn on a hand of a user and with the fingers of the user open, according to embodiments of the present technology;

FIG. 2 is the measurement device of FIG. 1 with the fingers of the user closed, according to embodiments of the present technology;

FIG. 3 is a top plan view of a measurement device when worn on a hand of a user and with the index finger of the user in abduction according to other embodiments of the present technology;

FIG. 4 is a top plan view of a first and a second finger connector of the measurement device of FIGS. 1 and 3, according to embodiments of the present technology;

FIG. 5 is a perspective view from the front of the measurement device of FIG. 3 with the middle finger of the user in flexion, according to embodiments of the present technology;

FIG. 6 is a perspective view from a top side of a measurement device depicting a guiding member and the first and the second finger connectors of FIG. 4, according to embodiments of the present technology;

FIG. 7 is a perspective view from a top side of the measurement device depicting an alternative embodiment of the guiding member of FIG. 6, according to embodiments of the present technology;

FIG. 8 is a perspective view from a top side of the measurement device depicting an alternative embodiment of the guiding member of FIG. 6, according to embodiments of the present technology;

FIG. 9 is a perspective view from a top side of the measurement device depicting an alternative embodiment of the guiding member of FIG. 6, according to embodiments of the present technology;

FIG. 10 is a perspective view from a bottom side of a measurement device depicting an interdigit guiding member, according to embodiments of the present technology;

FIG. 11 is the measurement device of FIG. 10 depicting an alternative embodiment of the interdigit guiding member, according to embodiments of the present technology;

FIG. 12 is a side view of a locking mechanism of a measurement device, according to embodiments of the present technology;

FIG. 13 is a perspective view from a front side of a measurement device depicting a clipper, according to embodiments of the present technology;

FIG. 14 is a perspective view from a top side of the measurement device of FIG. 10 before a string is released, according to embodiments of the present technology;

FIG. 15 is the measurement device of FIG. 14 depicting the release of the string, according to embodiments of the present technology;

FIGS. 16 and 17 are bottom views of a user's hand indicating measurements obtained for calibrating a measurement device, according to embodiments of the present technology;

FIGS. 18 and 19 are perspective views from a bottom side of the measurement device of FIG. 13 depicting a method of use of the measurement device, according to embodiments of the present technology;

FIG. 20 is a flow chart depicting a method of use for the measurement device, according to embodiments of the present technology;

FIG. 21 is a perspective view from a top side of a measurement device when worn on a hand of a user and with the fingers of the user open, according to other embodiments of the present technology;

FIG. 22 is the measurement device of FIG. 21 with the fingers of the user closed, according to embodiments of the present technology;

FIG. 23 is a perspective view from a bottom side of a measurement device when worn on a hand of a user and with the fingers of the user open, according to embodiments of the present technology.

FIG. 24 is a cross-section of a first finger of a measurement device, according to embodiments of the present technology;

FIG. 25 is a cross-section of a first finger of a measurement device, according to embodiments of the present technology;

FIG. 26 is a side view of an interdigit guiding member, according to embodiments of the present technology;

FIG. 27 is a top plan view of an interdigit guiding member according to embodiments of the present technology; and

FIG. 28 is a perspective view from a bottom side of a measurement device having the interdigit guiding member of FIG. 27, according to embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remain within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future.

With these fundamental principles in place, we will now consider some nonlimiting examples to illustrate various implementations of aspects of the present disclosure.

From a broad aspect, there is provided a measurement device for obtaining measurements of an anatomical space, such as but not limited to gynecological measurements of a vaginal canal or cervix during a pelvic examination. For the avoidance of doubt, pelvic examination broadly means an examination of all or a combination of the external and internal pelvic organs, such as but not limited to: vagina, cervix, vulva, uterus, fallopian tubes, ovaries, rectum as well as pelvic bones and muscles. For example, the measurement device can be used to collect information about at least some portions of the vaginal canal, vaginal width, the cervix, the uterus, pelvic bones, pelvic floor, and pelvic muscles. Examples of how these measurements can be used include: fitting gynecological implants; developing personalized gynecological implants, measuring dilation and/or effacement of the cervix during labor, and obtaining data about gynecological health. Gynecological implants may include pessaries for treating pelvic organ prolapse, urinary/fecal incontinence, and cervical incompetence. In other instances, embodiments of the measurement tool can be used for other anatomical cavities such as the anus, the mouth, the ear canal, the nostrils, etc.

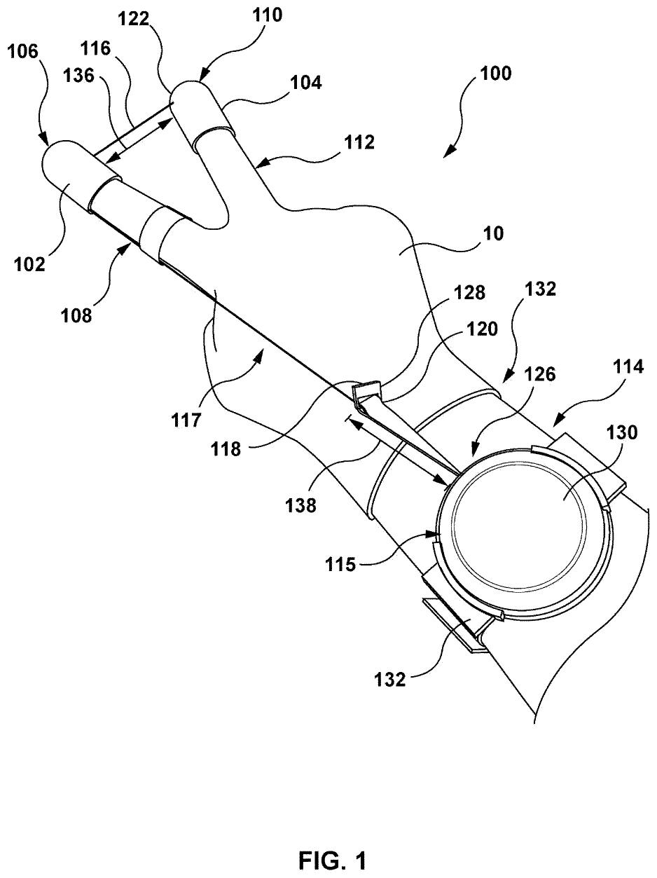

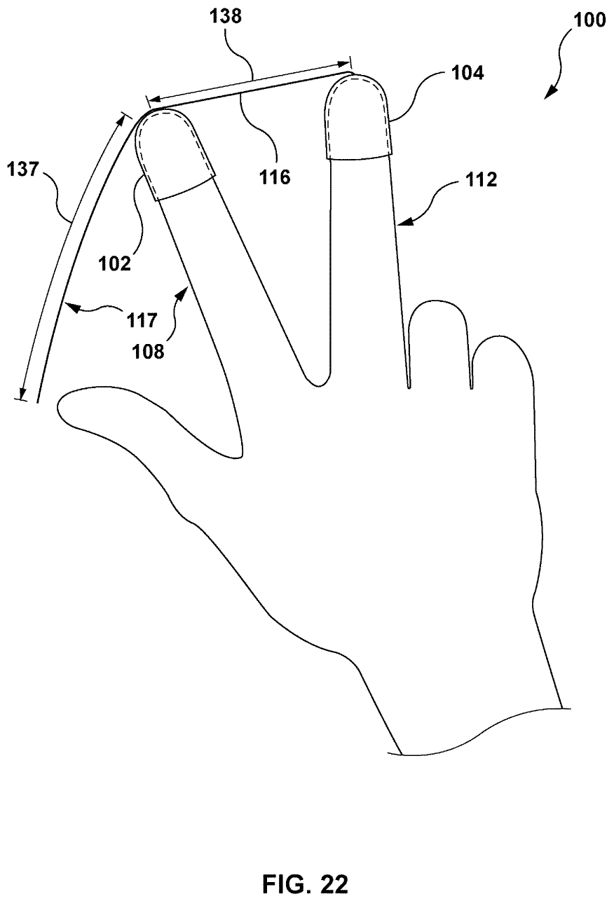

Broadly, referring to FIGS. 1 to 19, 21, and 22, there are depicted various embodiments of a measurement device 100 for obtaining measurements. The measurement device 100 includes a first finger connector 102 configured to connect to a first finger of a user and a second finger connector 104 configured to connect to a second finger of the user. A string 116 extends between the first finger connector 102 and the second finger connector 106 with an extension portion 117 extending beyond the first finger connector 102 by an extension length 137. One end of the string 122 is anchored to the second finger connector 104 and the string 116 is slidingly connected to the first finger connector 102.

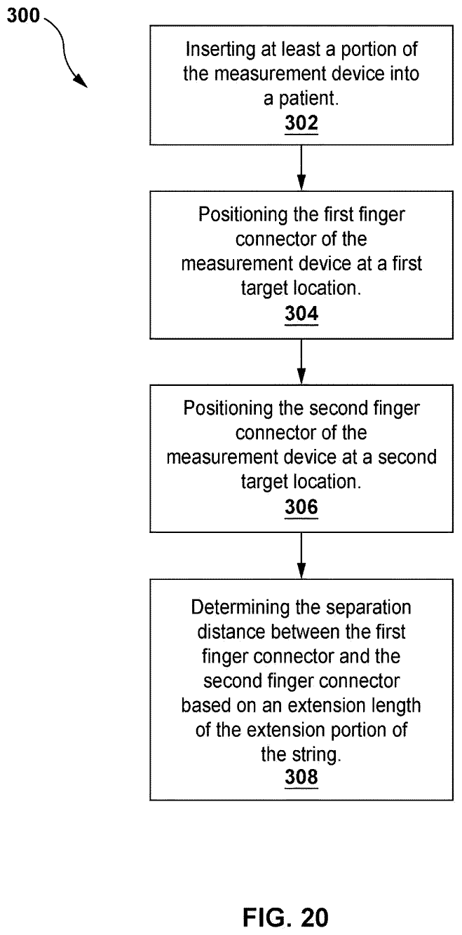

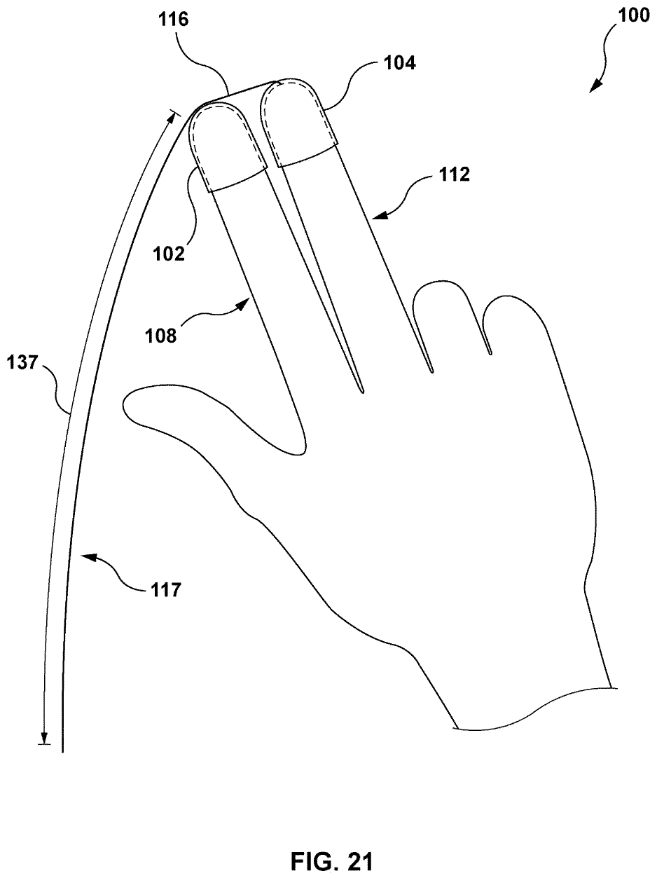

As the second finger connector 104 separates from the first finger connector 102, a separation distance 136 between the first and second finger connectors 102, 104 is modulated. In turn, the extension length 137 of the extension portion 117 of the string 116 is also modulated. The modulated separation distance 136 and the modulated extension length 137 are proportional to one another. In other words, a delta of the extension length 137 is proportional to a delta of the separation distance 136 as the first and second finger connectors 102, 104 move relative to each other. Therefore, the extension length 137 can be used to extrapolate the separation distance 136 and hence the measurement being taken between the first and second finger connectors 102, 104. More specifically, with reference to FIGS. 20 and 21, as the separation distance 136 increases, the extension length 137 decreases. The proportionality of the separation distance 136 increase and the extension length 137 decrease may be direct proportionality of 1:1.

An indicator 114 is provided for indicating a measure of the extension length 137 and/or the separation distance 136. As will be explained in further detail below, the measurement device 100 is configured such that when the first and second connectors 102, 104 are inserted into the anatomical space, such as the vaginal canal, the indicator 114 remains outside of the anatomical space and permits the measurements to be readily obtained and extracted.

### Fingers

In some embodiments, the first finger is an index finger, and the second finger is a middle finger. However, it is contemplated that the first and the second finger connectors may be connected to different fingers. Furthermore, the first and the second finger connectors do not need to be placed on fingers adjacent to one another.

It is further contemplated that the measurement device 100 may be configured for a left hand or a right hand of the user. Furthermore, the measurement device 100 may be configured to be ambidextrous, in that features of the measurement device 100 may be centralized such that the same measurement device 100 accommodates both the left or right hand.

The term “fingers” should be interpreted in a broad sense, including human fingers such as of the practitioner taking the measurements, or of the user taking measurements of themselves. The term “fingers” is also intended to cover prosthetic fingers, robotic fingers, or finger-like digits of any form or configuration that can be moved relative to each other.

The term “connector” should also be interpreted in a broad sense and is intended to include embodiments in which the finger connector is not intended for connection to a finger but can be a member operable independently. For example, the finger connectors may be configured as digits movable relative to each other, the control of which can be operated by a user externally to the patient.

### String

The string 116 is composed of a biocompatible material, such as a suture string. The string 116 has properties enabling accurate measurement such as tensile properties appropriate to the tension applied when the first and second finger connectors 102, 104 are moved apart. In some embodiments, the string 116 is made of one or more of: nylon, polypropylene, silk, cotton, and polyester. The string 116 may have a monofilament or a braided configuration. The string 116 may have a circular profile, or have a flattened profile such as a tape. In some embodiments, the string 116 is a suture string made of poly (p-dioxanone) which is absorbable and a monofilament (e.g. PDS II™). The string 116 may include the indicator 114 thereon, as will be described below. The anchoring of the string 122 to the second finger connector 104 can be by any means, such as a knot, biocompatible glue, or a combination thereof.

The string 116 has another end 118, which may be a free end, or be connected to the indicator 114, as will be described below.

### Indicator

The indicator 114 can be any type of indicator of a measurement value of the extension length 137 and/or the separation distance 136. As noted above, the indicator 114 is configured to be accessible from outside of the anatomical cavity when the measurements are taking place.

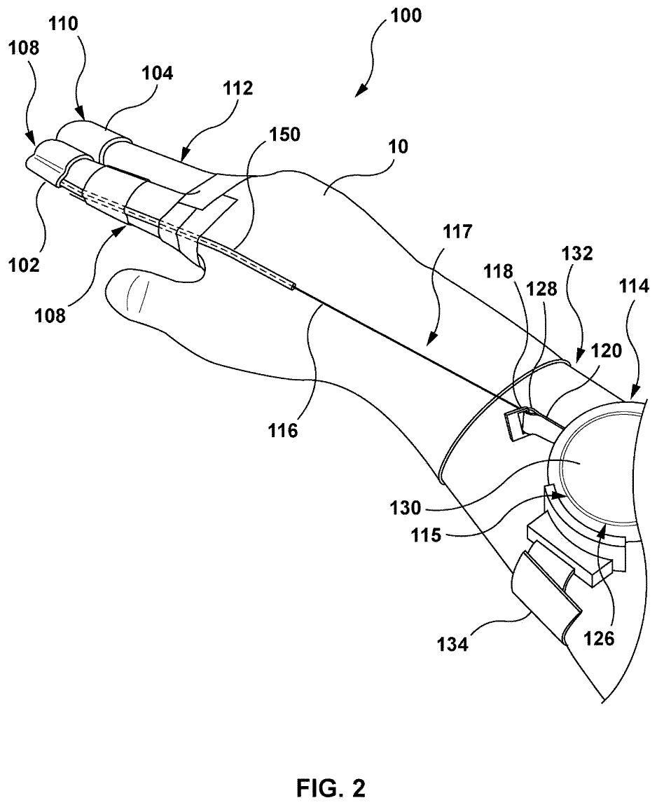

In the embodiments depicted in FIGS. 1 and 2, the indicator 114 is a spool assembly 115 including a spooled tape measure 126 that can extend out of and retract into a body 130 of the spool assembly 115. The spool assembly 115 is retractable, such that the tape measure 126 is either biased to a retracted configuration by means of a spring (not shown), or can be retracted from an extended configuration by an actuator such as a button. The spool assembly 115 is positionable on a wrist 132 of the user.

In this respect, in certain embodiments, the body 130 of the tape measure 126 is mounted to a strap 134 which is placed around the wrist 132 of the user. The strap 134 may include a fastener such as a hook-and-loop fastener, a buckle, a clasp, or a combination thereof, which allows for adjustment and securement of the strap 134 to the wrist 132. In other embodiments, the spool assembly 115 is positionable on any other body part of the user, or to a workstation of the user. An end 120 of the tape measure 126 includes an attachment feature 128, such as a hood or a hook, to which the end 118 of the string 116 may be connected.

The string 116 extends between the end 120 of the tape measure 126 and slidably connects with the first finger connector 102. The string 116 extends between the first finger connector 102 to the second finger connector 104, where the other end 122 of the string 116 is anchored to the second finger connector 104, such that the string 116 is slidingly moveable with respect to the first finger connector 102.

As the first finger connector 102 and the second finger connector 104 are moved apart to make a measurement, the separation distance 136 increases, in turn the extension length decreases, and causes the tape measure 126 to extend from the body 130 by a spooled length 138. The modulation of the separation distance 136 is proportional to the spooled length 138, as well as the extension length 137.

The spooled length 138 can be determined from markings on the tape measure. Thus, the user is informed of the separation distance 136 between the first and the second finger connectors 102, 104 by the spooled length 138 of the tape measure 126. The type of markings is not limited and may comprise numbers, colours, other visual indicia, tactile indica, etc.

In use, the first and second finger connectors 102, 104 are inserted into the anatomical cavity and positioned relative to each other in order to take a desired measurement within the anatomical cavity. Generally, as it is easier to insert the first and second finger connectors 102, 104 whilst they are closer together, typically the first and second finger connectors 102, 104 are inserted into the anatomical cavity whilst close together then moved apart from each other for the measurement. The first and second finger connectors 102, 104 are positioned at the extremities of the distance to be measured (first and second target locations). Thus, the separation distance 136 between the first and second finger connectors 102, 104 is the distance to be measured (between the target locations). The user would determine the separation distance 136 based on the spooled length 138.

It is contemplated, in alternative embodiments, that the indicator 114 may be a different type of spool assembly. For example, instead of the spool assembly 115 having a measuring tape 126, it is the extension portion 117 of the string 116 that is spooled. In other words, the string 116 extends and retracts from the spool assembly 115. In such embodiments, similar to what has been previously described, as the first and the second finger connectors 102, 104 are moved apart from one another by a separation distance 136, the string 116 spools from the spool assembly 115 by a spooled length 138 which is proportional to the separation distance 136. The extension portion 117 of the string 116 may include any type of markings to enable the measurement of the spooled length 138 or the extension length such as, but not limited to, colours, numbers, letters, other visual indicia, tactile indicia, and the like.

In other embodiments, the markings representative of the measurement of the spooled length 138 or the extension portion may be presented on the body 130 or other part of the spool assembly 115.

In certain embodiments of any of the above, the indicator 114 may be configured to produce a digital output of the spooled length 138. For example, the indicator 114 may be configured as a screen to display the digital output. In some instances, the screen may be mountable to the user, for example the wrist 132 or forearm. In another instance, the screen may be configured to be detachable from the measurement device 100.

It is appreciated that the spool assembly 115 as described may be removably connected to the end 118 of the extension portion 117 permitting the spool assembly 115 to be disconnected from the measuring device, sterilized and reused for multiple procedures (such as by re-attaching to the string 116 of a fresh measurement device 100). In alternative embodiments, the measurement device 100 may be fully disposable.

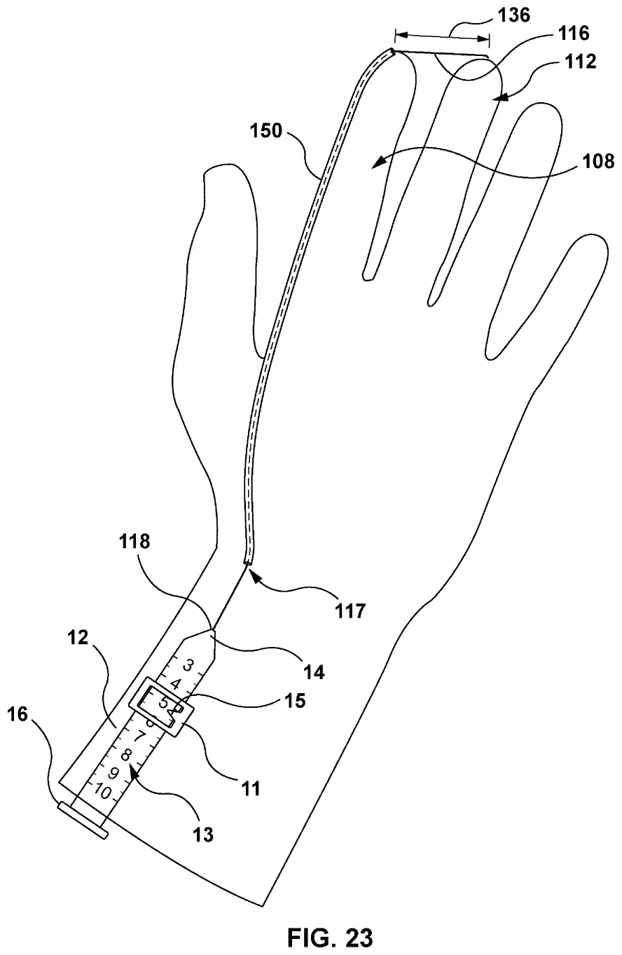

As depicted in FIG. 23, in further alternative embodiments, the indicator 114 may comprise a window 11 and a tab 12 having markings 13 (e.g., colours, indicia, letters, numbers, and/or tactile markings) disposed along a length of said tab 12. The window 11 is fixed while the tab 12 is configured to slide relative to the window 11. One end 14 of the tab 12 is connected to the end 118 of the extension portion 117. Similar to what has been previously described, as the first and second finger connectors 102, 104 are moved apart from one another by the separation distance 136, the tab 12 slides relative to the window 11. The window 11 may further include a pointer 15 which the user may reference to read the marking 13 of the tab 12 which is proportional to the separation distance 136. In certain embodiments, the other end of the tab 12 includes a stopper portion 16 which prevents further sliding of the tab 12 relative to the window. In other words, the stopper portion 16 stops the tab 12 from sliding out completely.

It is contemplated, in alternative embodiments, the window 11 and the tab 12 may be fixed. In this embodiment, the end 118 of the extension portion 117 may be connected to the point 15 such that the pointer 15 slides relative to the window 11 and the tab 12.

In yet further alternative embodiments, the spool assembly 115 may be omitted. In such embodiments, the end 118 of the extension portion 117 of the string 116 may be a free end, connected to a pull tab, or be connected to something else. The indicator 114 may comprise visual markings (e.g., colours, indicia, letters, or numbers) and/or tactile markings disposed on the string 116, for example along the extension portion 117. As such, the extension length 137 of the extension portion 117 is proportional to the separation distance between the first and the second finger connectors 102, 104.

### Finger Connectors

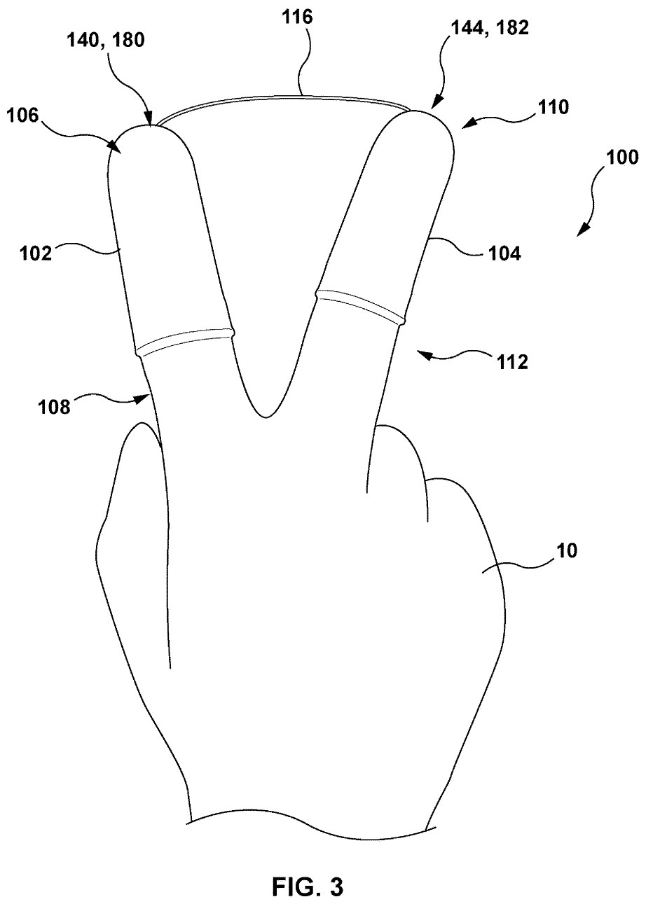

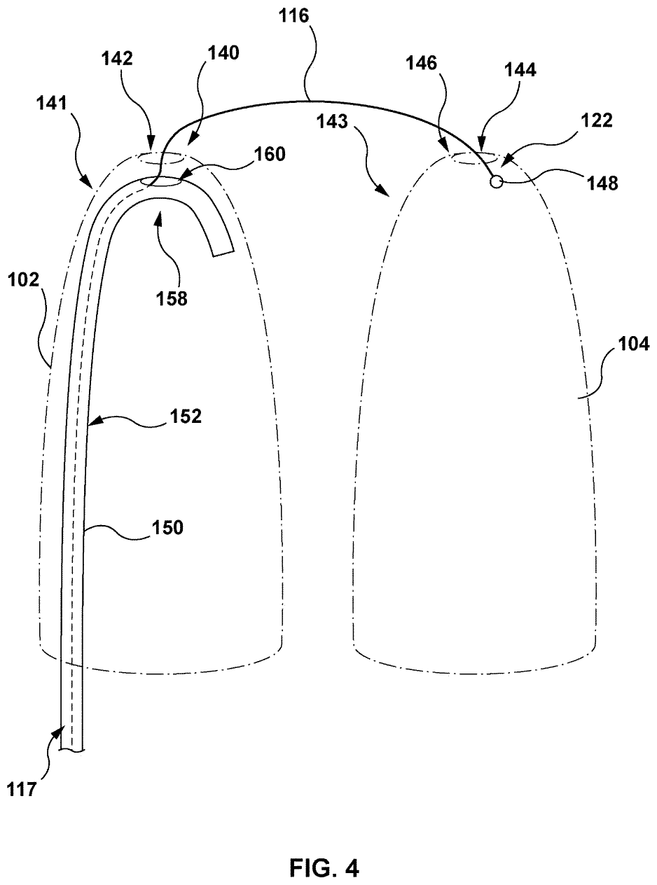

With specific reference to FIGS. 3 and 4, one or both of the first and second finger connectors 102, 104 may be configured as a cap. Each cap may be configured to accommodate a fingertip. For example, a first finger cap (corresponding to the first finger connector 102) may be configured to receive a fingertip 106 of the index finger 108. As another example, a second finger cap (corresponding to the second finger connector 104) may be configured to receive a fingertip 110 of and the middle finger 112. The caps are configured to provide a snug fit onto the respective fingertips. As depicted in the figures, the caps are configured to extend to proximal interphalangeal joints of the fingers on which they are worn. In other embodiments, the caps may have other sizes and configurations. For example, one or both of the caps may stop short of the proximal interphalangeal joints of the fingers and have a shallower profile. In another example, one or both of the caps may be ring-like. In further examples, one or both caps may be configured to partially cover the fingertip 106, 110 extending to proximal the interphalangeal joints of the finger on which they are worn. In some instances, the caps may be configured to not block portions of the fingertips 106, 110 so as to not impede tactile feedback. The first and the second finger connectors 102, 104 may be made of any suitable material such as latex, silicone, or any other biocompatible material.

As depicted in FIG. 4, the first finger connector 102 is configured to slidably connect with the string 116. In certain embodiments, the string 116 slidably connects with the first finger connector 102 at a center portion 140 of a distal tip 141 of the first finger connector 102. The first finger connector 102 includes an opening 142 disposed at the center portion 140 of the distal tip 141. The string 116 is disposed beneath the first finger connector 102 and passes through the opening 142, forming the slidable connection with the string 116. The string 116 being disposed beneath the first finger connector 102 prevents slippage or inadvertent movement of the string 116 during use of the measurement device 100.

The second finger connector 104 is configured to anchor the string 116 extending from the first finger connector 102. The second finger connector 104 includes an opening 144 disposed at a center portion 146 of a distal tip 147 of the second finger connector 104. The end 122 of the string 116 is passed through the opening 144 of the second finger connector 104 and disposed beneath the second finger connector 104 where it is anchored 148. In some embodiments, the distal end portion 122 is tied into a knot or glued to an inside surface of the second finger connector 104. The string 116 being secured beneath the second finger connector 104 prevents slippage or inadvertent movement of the string 116 during use of the measurement device 100. Furthermore, having the distal end portion 122 secured to the interior surface of the second finger connector 104 prevents accidental disconnection of the distal end portion 122 from the second finger connector 104.

The first and the second finger connectors 102, 104 provide a center-to-center configuration of the string 116. That is, the string 116 extends from the opening 142, disposed at the center portion 140 of the first finger connector 102, to the opening 144, disposed at the center portion 146 of the second finger connector 104, to provide center-to-center measurements. The center-to-center configuration avoids components of the measurement device 100 impeding tactile feedback during pelvic examinations. Specifically, disposing the strings 116 at the center portions 140, 146 of the distal tips 141, 147 of the first and the second finger connectors 102, 104 allow users to feel tactile feedback on finger pads of the index and middle fingers 108, 112 without any interference from components of the measurement device 100. Thus, users are able to palpate during the pelvic examination without interference from the measurement device 100.

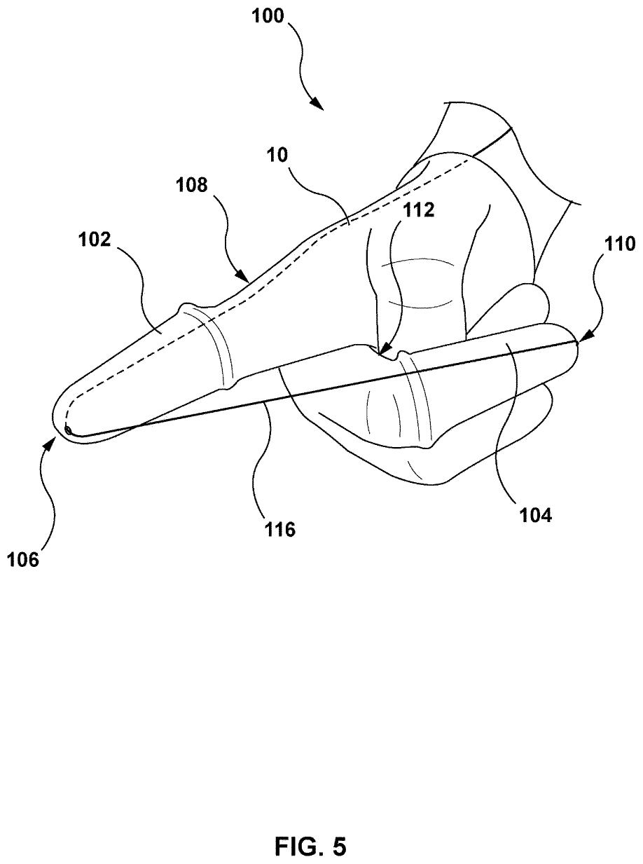

As depicted in FIG. 5, the center-to-center configuration provides users with a wider range of motion, a variety of angles, and accurate, reproducible measurements. For example, the first and second finger connectors 102, 104 can be moved apart on a same plane, that is to say the index finger 108 is in abduction (as shown in FIG. 3) or on different planes, such as the middle finger 112 being in flexion (as shown in FIG. 5). The center-to-center configuration allows for all types of measurement including, but not limited to, the distance between the ischial spines, between the ischial spines and coccyx, between the ischial spines and pubic symphysis, anterior and posterior fornices width, width of the upper vagina, mid vagina and low vagina, mid-lower and medial vagina, width of the medial vagina, and width of the exocervix and exocervical canal.

In yet other embodiments, instead of a cap, any other configuration of connector can be used to connect to a finger. In some embodiments (not shown), one or both of the first and the second finger connectors 102, 104 may be configured as a ring connectable to a fingertip. For example, each ring may be positionable on a respective finger such that the string 116 is positioned at the center portion of the distal tip.

In further embodiments, the first and second finger connectors 102, 104 may be integral with, or otherwise connected to a glove or other hand covering, we will be described in further detail below.

### Hand Covering

Referring to FIGS. 1 to 3, and 5, in certain embodiments, the measurement device 100 is configured as a hand covering such as a glove 10 with which the first and the second finger connectors 102, 104 disposed in relation to. The first and the second finger connectors 102, 104 may be integral with, or connected to the glove 10. By integral is meant that the first and second finger connectors comprise finger portions of the hand covering. In the integral embodiments, the glove and the finger connectors 102, 104 are one piece and produced integrally with one another. For example, the glove 10 may comprise reinforced portions at tips of certain fingers of the glove 10 that define the finger connectors 102, 104. In other embodiments of the integral glove 10/first and the second finger connectors 102, 104, the glove 10 may be layered and the first and second finger connectors 102, 104 may be positioned at any position with respect to the glove 10 layers. In certain embodiments, the glove 10 is composed of a hypo-allergenic material and is a sterile and powder-free surgical glove.

With reference to FIGS. 1 and 2, in certain embodiments, the glove 10 and the first and the second finger connectors 102, 104 are separate but can be assembled together. In such embodiments, the glove 10 defines a base layer of the measurement device 100. In other words, the glove 10 is disposed beneath the first and the finger second finger connectors 102, 104 and the string 116. In this embodiment, the first and the second finger connectors 102 are configured to receive an index finger 108 of the glove 10 and a middle finger 112 of the glove 10. The string 116 being secured beneath the second finger connector 104 prevents slippage or inadvertent movement of the string 116 during use of the measurement device 100, ensuring center-to-center measurements can be obtained. Furthermore, having the distal end portion 122 secured to the interior surface of the second finger connector 104 prevents accidental disconnection of the distal end portion 122 from the second finger connector104.

As depicted in FIGS. 3 and 5, in certain embodiments, the glove 10 and the first and the second finger connectors 102, 104 are separate but can be assembled together. In such embodiments, the glove 10 provides a top layer of the measurement device 100. The glove 10 is disposed atop the first and the second finger connectors 102, 104, and the string 116. In other words, the index finger 108 of the glove 10 houses the first finger connector 102 and the middle finger 112 of the glove 10 houses the second finger connector 104. In this embodiment, the glove 10 includes an opening 180, 182 disposed on each of the tips of the index finger 108 of the glove 10 and the middle finger 112 of the glove 10, respectively. The openings 180, 182 align with the center portion of the distal tip 140, 144 of each of the first and the second finger connectors 102, 104. The string 116 passes through the opening 180 of the glove 10 on the index finger 108 of the glove 10, extends towards the middle finger 112 of the glove 10, passing through the opening 182 of the glove 10 of the middle finger 112 of the glove 10.

In some embodiments, the end 122 of the string 116 may be anchored onto the glove 10. The string 116 being anchored onto the glove 10, prevents slippage or inadvertent movement of the string 116 during use of the measurement device 100, ensuring center-to-center measurements can be obtained. For example, the end 122 of the string 116 may be directly tied onto the glove 10. In another embodiment, the end 122 of the string 116 may be anchored beneath the glove 10. For example, the end 122 may be anchored by a knot disposed under the glove 10, directly anchored onto the glove 10, directly anchored to an interior or exterior surface of the second finger connector 104, or a combination thereof. In a further alternative embodiment, the end 122 may be anchored at another location on the glove 10 or a different component of the measurement device 100. For example, the end 122 may be anchored onto a wrist portion of the glove 10 or may be anchored onto the indicator 114 (e.g., the strap 134). It is further contemplated that the end 122 may be anchored separate from the measurement device 100.

It is contemplated, in alternative embodiments, that the measurement device 100 may include two gloves. A first glove is used as the base layer and a second glove is disposed overtop.

It is further contemplated, in alternative embodiments, that the measurement device 100 may be configured as a portion of the glove 10, that is a partial glove. For example, the measurement device 100 may comprise the first finger (e.g., the index finger 108) and the second finger (e.g., the middle finger 112) of the glove 10. In some instances, the measurement device 100 may further include a third finger (e.g., a thumb) of the glove 10. In some instances, the measurement device 100 may further include interdigit guiding members, locking mechanisms, and/or glove measurement markings which will be described in further detail below. The partial glove 10 may be configured to slide overtop of a user's hand and/or overtop of a regular glove.

### Guiding Member

As can be seen most clearly in FIG. 4, and 6 to 9, the measurement device 100 includes a guiding member 150 configured to receive and guide a relative movement of the extension portion 117 of the string 116. The guiding member 150 can have any configuration which can provide a pathway along which the string 116 will move. In some embodiments, the guiding member 150 is a tube 150 having a channel for receiving the extension portion 117. The tube 150 guides the string 116 movement and prevents slippage of the string 116, thereby improving an accuracy of the measurements as the string is guided in its movement. Additionally, the tube 150 prevents the string 116 from being pinched or snagged during use, thereby providing a smooth motion for the user.

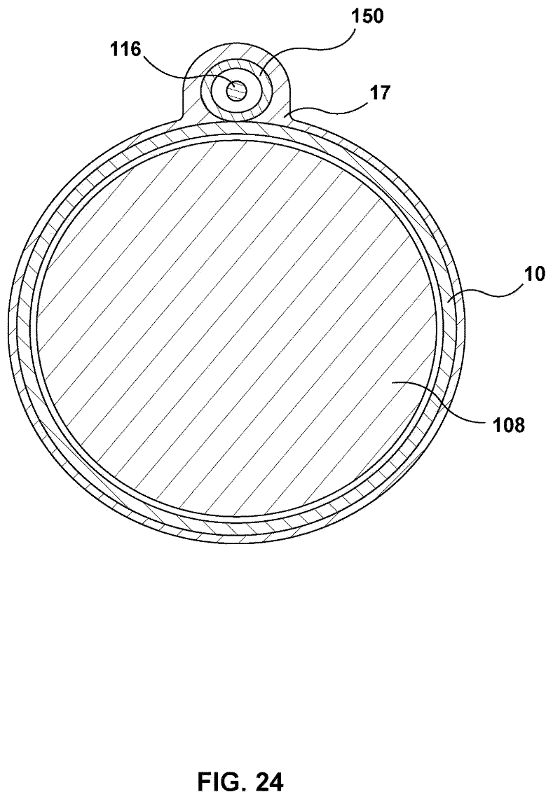

The tube 150 is attached to the glove 10, such as by one or more of medical tape, biocompatible glue, or a combination thereof. As depicted in FIG. 2, the tube 150 may be attached to the exterior surface of the glove 10 (that is, when the glove 10 is disposed beneath the first and the second finger connectors 102, 104). As depicted in FIGS. 3 and 5, the tube is attached to the interior surface of the glove 10 (that is, when the glove 10 is disposed over top the first and the second finger connectors 102, 104). It will be appreciated that the guiding member 150 may also be integral with the glove 10 or one or both of the first and second finger connectors 102, 104. With reference to FIG. 24, in certain embodiments, the glove 10 and the attached tube 150 may be dipped in a polymer or a rubber, such as a nitrile (for example) to form an outer layer 17, encompassing the tube 150 and glove 10.

With reference to FIG. 4, the tube 150 is disposed within the first finger connector 102. In some embodiments, a first end portion 152 of the tube 150 follows a contour of the distal portion of the finger connector 102. The first end portion defines an arc 158. When the measurement device 100 is worn by the user, the arc 158 is positioned over the fingertip 106 of the index finger 108. In certain embodiments, the first end portion 152 of the tube 150 terminates past the center portion 140 of the first finger connector 102. The arc 158 of the tube 150 includes an opening 160 which aligns with the opening 142 of the first finger connector 102. The string 116 passes through the opening 160 of the tube 150, exiting out of the first finger connector 102 from the opening 142. The opening 160 helps to fix a position of the string 116 relative to the finger connector 102, thereby also providing more accurate measurements.

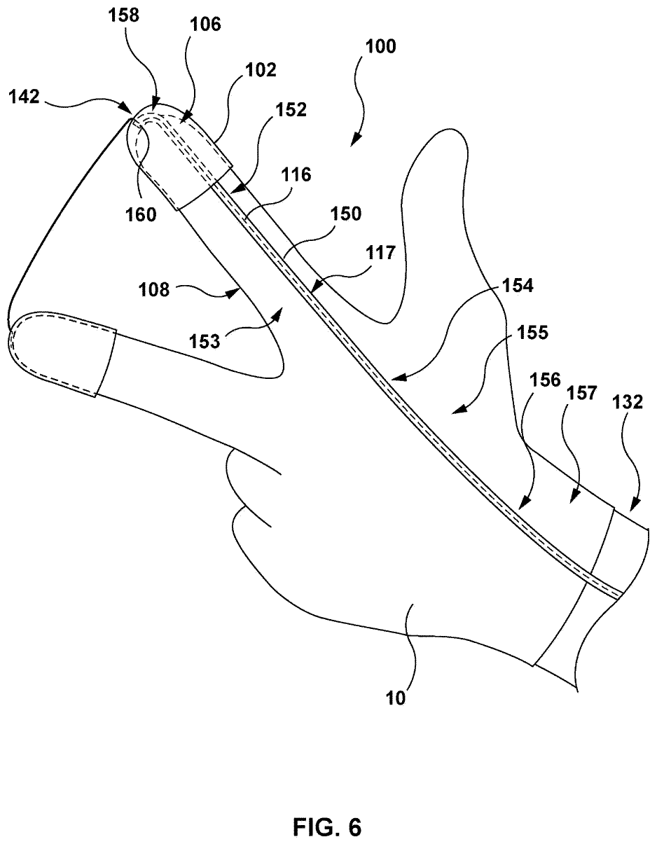

As depicted in FIG. 6, in certain embodiments, the tube 150 extends between the wrist 132 to the first finger connector 102. The tube 150 is connected to the glove 10 such that the first end portion 152 of the tube 150 is disposed along a portion 153 of the index finger 108 of the glove 10, an intermediate portion 154 of the tube 150 is disposed along a top side 155 of the glove 10, and a second end portion 156 of the tube 150 is disposed along a wrist portion 157 of the glove 10. In some embodiments, the first end portion 152 may be disposed along an outer edge 157 of the index finger 108 of the glove 10. The intermediate portion 154 may be disposed along a top side 155 of the glove 10. The second end portion 156 may be disposed along the wrist portion 157 of the glove 10. Thus, the measurement device 100 is ambidextrous as the glove 10 can be worn on either the right hand or the left hand of the user. In other words, the tube 150 can be positioned centrally between palmer and dorsal portions of the glove 10 enabling the glove 10 to be worn on a left or right hand of the user. In yet other embodiments, the tube 150 can be positioned along a palm side of the glove or a dorsal side of the glove. In those embodiments, the glove is specifically configured for the left hand or the right hand.

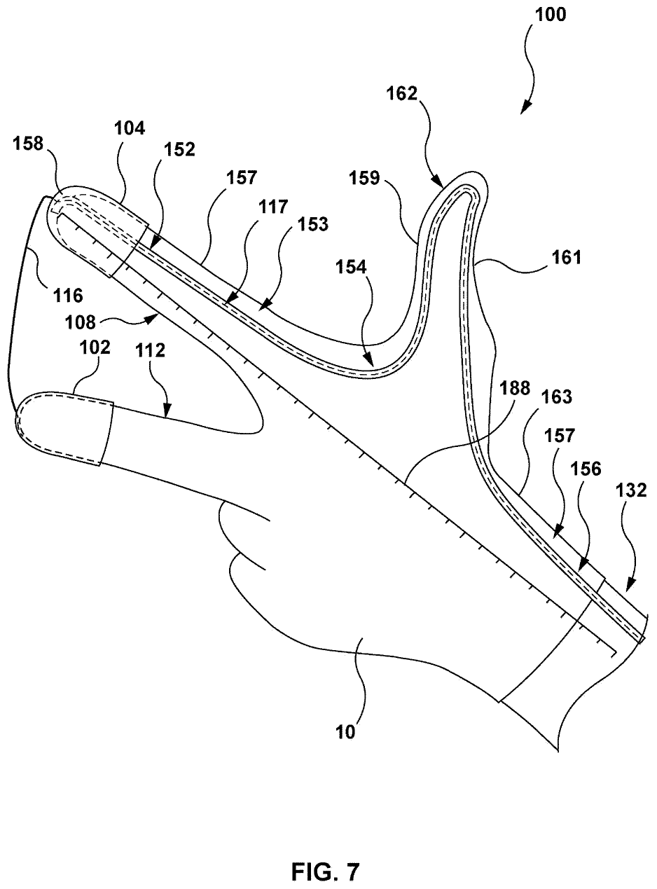

As shown in FIG. 7, in other embodiments, the intermediate portion 154 of the tube 150 is disposed along a third finger 162 of the glove 10, for example for housing a thumb 162 of the user. In this instance, the first end portion 152 of the tube 150 is disposed along a portion 153 of the index finger 108 of the glove 10, the intermediate region 154 is disposed along a portion of the thumb 162 of the glove 10, and the second end portion 156 is disposed along a wrist portion 157 of the glove 10. Specifically, the intermediate portion 154 includes a portion which is disposed on an inner edge 159 of the thumb 162 of the glove 10 and another portion which is disposed on an outer edge 161 of the thumb 162 of the glove 10. In some embodiments, the first end portion 152 may be disposed along an outer edge 157 of the index finger 108 of the glove 10 and the second end portion may be disposed along an outer edge 163 of the wrist portion 157. Thus, the measurement device 100 is ambidextrous as the glove 10 can be worn on either the right hand or the left hand of the user.

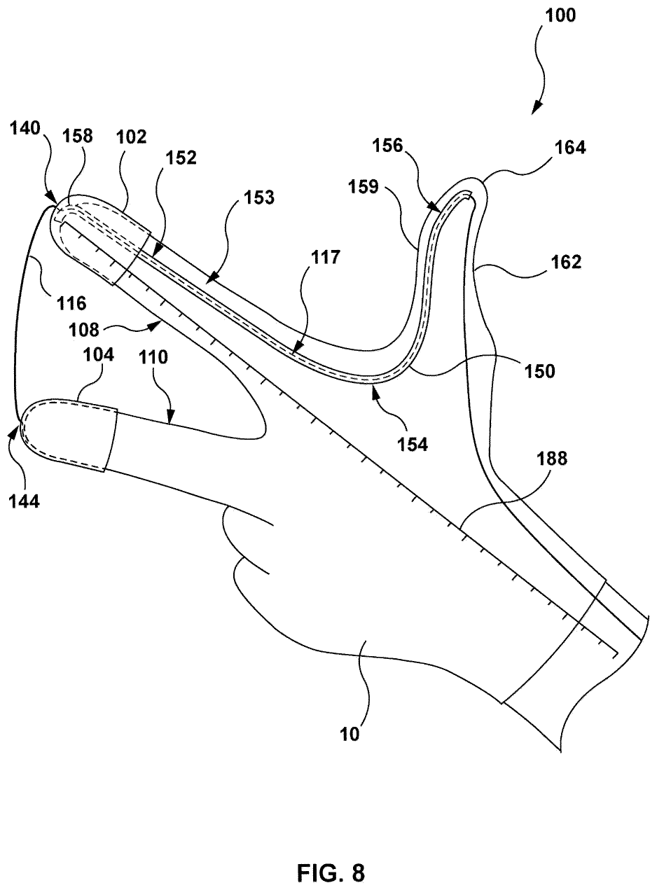

With reference to FIG. 8, a further alternative embodiment of the arrangement of the tube 150 is depicted. In this embodiment, the first end portion 152 of the tube 150 is disposed along the portion 153 of the index finger 108 of the glove 10 and the second end portion 156 is disposed along the portion of the thumb 162 of the glove 10 with the intermediate portion 154 of the tube 150 extending therebetween (e.g., along a pericule of the glove 10) The second end portion 156 of the tube 150 terminates at a fingertip 164 of the thumb 162 of the glove 10 and the string 116 extends towards the wrist 132 of the user. In some embodiments, the first portion 152 is disposed along the outer edge 157 of the index finger 108 of the glove 10 and the second portion 156 is disposed along the inner edge 159 of the thumb 162 of the glove 10. Thus, the measurement device 100 is ambidextrous as the glove 10 can be worn on either the right hand or the left hand of the user.

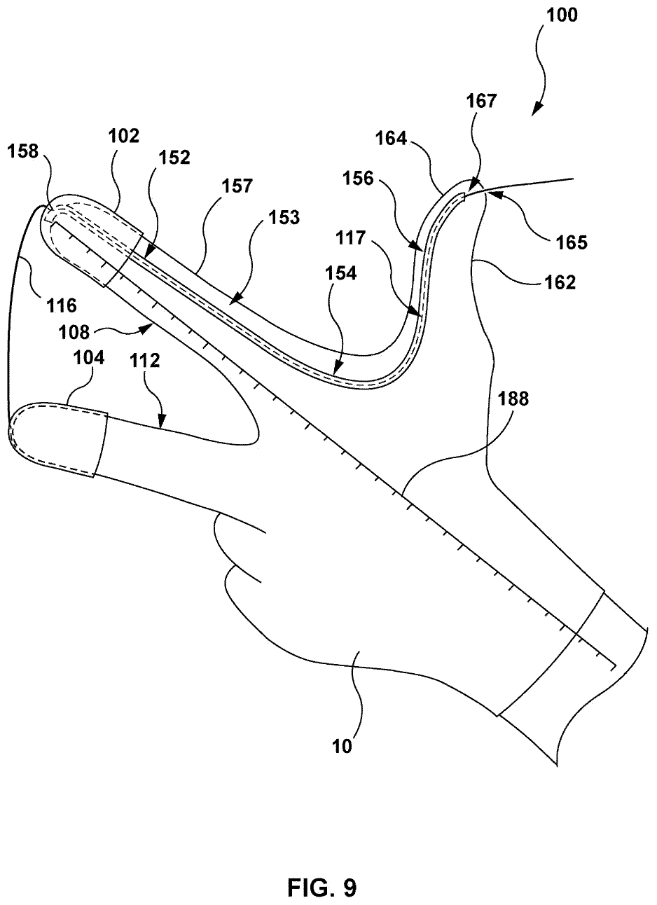

Referring to FIG. 9, a further alternative embodiment of the arrangement of the tube 150 is depicted. In this embodiment, the string 116 and/or tube 150 do not extend towards the wrist 132 of the user. Instead, the first end portion 152 of the tube 150 is disposed along the portion 153 of the index finger 108 of the glove 10 and the second end portion 156 is disposed along the portion of the thumb 162 of the glove 10 with the intermediate portion 154 of the tube 150 extending therebetween (e.g., along the pericule of the glove 10) The second end portion 156 of the tube 150 terminates at a fingertip 164 of the thumb 162 of the glove 10. The thumb 162 of the glove 10 includes an opening 165 aligned with an opening 167 of the second end portion 156 of the tube 150, thereby allowing the string 116 to extend away from the thumb 162. In some embodiments, the first portion 152 is disposed along the outer edge 157 of the index finger 108 of the glove 10 and the second portion 156 is disposed along the inner edge 159 of the thumb 162 of the glove 10. Thus, the measurement device 100 is ambidextrous as the glove 10 can be worn on either the right hand or the left hand of the user.

In some embodiments, the tube 150 and/or the string 116 is pre-lubricated to decrease friction between the tube 150 and the string 116, thus providing a smooth movement of the string 116 within the tube 150. In some embodiments, the tube 150 may include an injection opening (not shown) in which the user may inject a lubricant to improve the slidability of the string 116 within the tube 150.

It is contemplated, in alternative embodiments, that the tube 150 may be disposed in a different arrangement. For example, instead of a continuous tube 150, the measuring device 100 may include a plurality of tubes 150 separated from one another, each configured to receive a segment of the string 116. In a specific example, there may be three tubes 150 with each tube 150 corresponding to the first end portion 152, the intermediate portion 154, and the second end portion 156. It is further contemplated that the tube 150 may be disposed along other portions of the glove 10.

Although the tube 150 is shown as a separate component attached to the glove 10, it is appreciated that at least some of the tube 150 may be formed integrally with the glove 10.

In other embodiments, the guiding member 150 may be provided without the glove 10. In such examples, the guiding member 150 will be connected to one or both of the first and second finger connectors 102, 104.

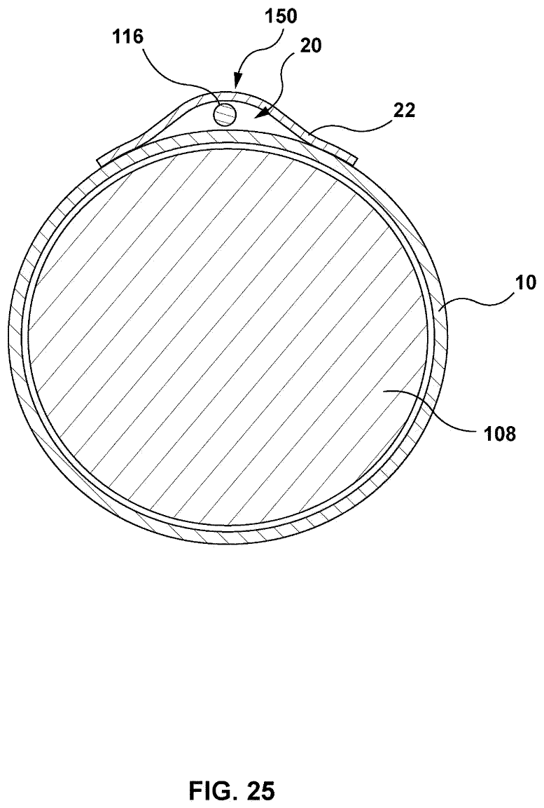

In alternative embodiments, the guiding member 150 may comprise a channel 20 formed by attaching an arched member 22 along an outer surface of the glove 10 (FIG. 25). The arched member 22 may be formed from a flat sheet that is subsequently curved. It is contemplated that the channel 20 may assume any shape configured to receive the extension portion 117 of the string 116. In alternative embodiment, the channel 20 may formed by another layer with the same or different material. The additional layer may be formed in any manner such as by dipping.

### Interdigit Guiding Member

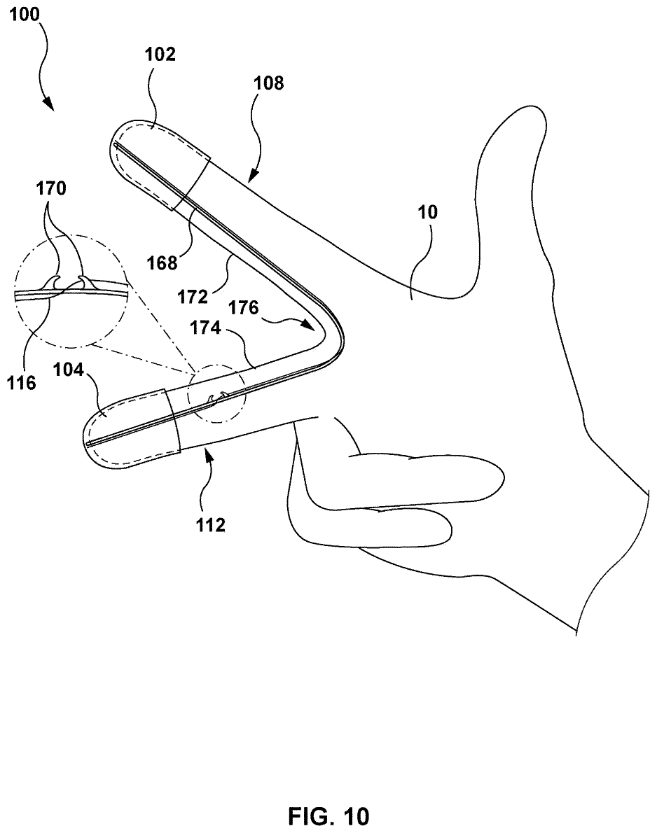

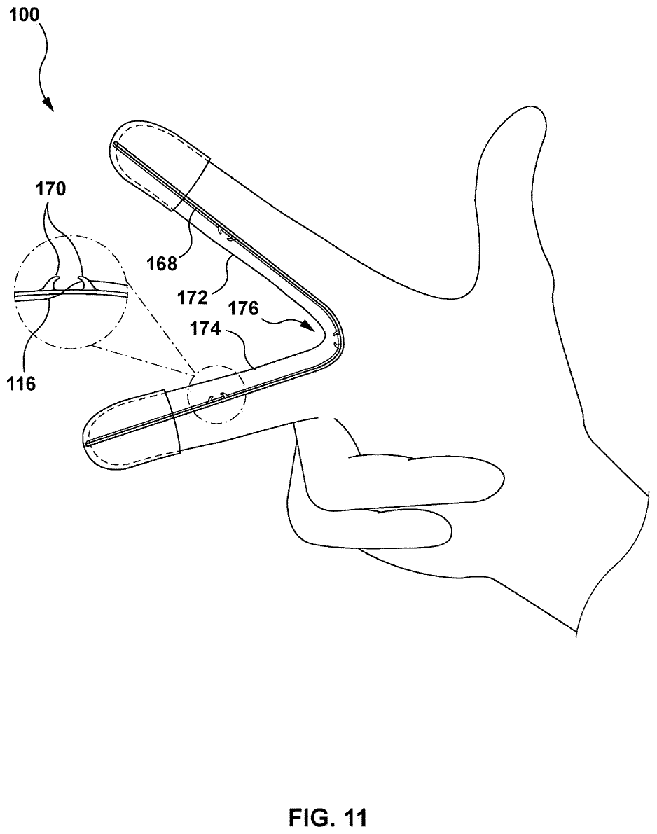

With reference to FIGS. 10 and 11, in certain embodiments, the measurement device 100 includes an interdigit guiding member 168 disposed in between the first finger connector 102 and the second finger connector 104. The interdigit guiding member 168 is continuous between the first finger connector 102 and the second finger connector 104. In certain embodiments, the interdigit guiding member 168 is disposed along an inner edge 172 of the index finger 108 of the glove 10 and an inner edge 174 of the middle finger 112 of the glove 10, extending between an interdigit 176 of the index finger 108 of the glove 10 and the middle finger 112 of the glove 10. The interdigit guiding member 168 is disposed relative to the tube 150, such that the interdigit guiding member 168 does not interfere with the positioning of the tube 150. For example, the interdigit guiding member 168 may be disposed on a bottom side of glove 10, while the tube 150 is disposed on the top side of the glove 10. Alternatively, the interdigit guiding member 168 may be disposed such that it is positioned beside the tube 150.

With reference to FIGS. 10 and 11, in certain embodiments, at least a portion of the interdigit guiding member 168, includes retention arms 170 for releasably retaining the string 116. The interdigit guiding member 168 retains the string 116 to guide the string 116 and enable easier string 116 movement (such that the string 116 does not get caught or snagged during insertion of the measurement device 100). Additionally, retaining the string 116 prior to measurements provides users with the ability to perform portions of the pelvic examination without interference from the string 116 (for example, when palpating locations within the vaginal canal). The user, thus, can perform portions of the pelvic exam while the string 116 is retained and deploy the string 116 when required.

As depicted in FIG. 10, the retention arms 170 extend an entire length of the interdigit guiding member 168. Alternatively, as depicted in FIG. 11, the interdigit guiding member 168 may include discrete retention arms 170 that are distributed along the interdigit guiding member 168. For example, the interdigit guiding member 170 includes three discrete retention arms 170, spaced apart from one another, distributed along the length of the interdigit guiding member 170.

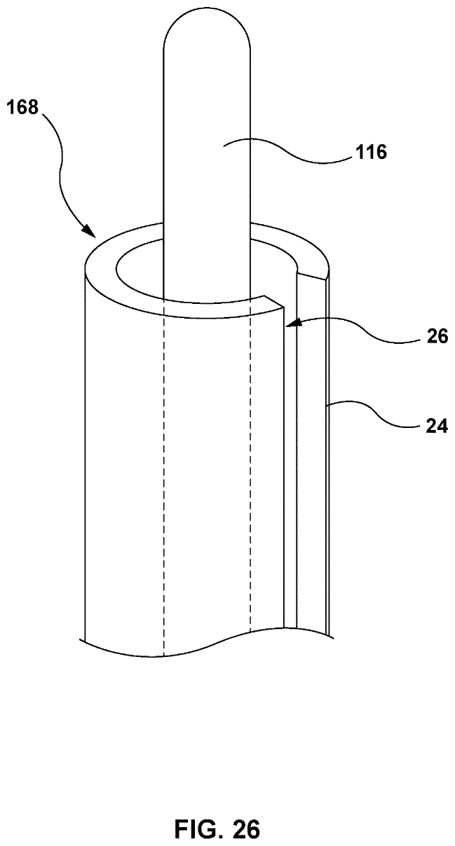

An alternative embodiment of the interdigit guiding member 168 is depicted in FIG. 26. The interdigit guiding member 168 may include a tube 24 in which a slit 26 is disposed along a length of said tube 24. When not in use, the string 116 is held within the tube 24. When the user is ready to perform the measurement, the string 116 is released from the tube 24 via the slit 26.

In some embodiments, in which the glove 10 is disposed beneath the first and the second finger connector 102, 104, the interdigit guiding member 168 is attached onto the exterior of the first finger connector 102, the second finger connector 104, and the exterior of the glove 10 (that is, along the interdigit portion 176 of the glove 10). The interdigit guiding member 168 extends from the center portion 140 of the first finger connector 102 to the center portion 146 of the second finger connector 104. That is, the interdigit guiding member 168 is positioned proximate to the openings 142, 144 of the respective first and the second finger connectors 102, 104, such that the retention arms 170 immediately releasably retain the string 116 outside of the openings 142, 144.

It is contemplated that, in an alternative embodiment, the interdigit guiding member 168 may not be positioned from the center portion 140 of the first finger connector 102 to the center portion 146 of the second finger connector 104. For example, the interdigit guiding member 168 may be disposed at a bottom portion (not shown) of the first finger connector 102 and the second finger connector 104. Alternatively, in an alternative embodiment, the interdigit guiding member 168 may be positioned at the center portion 140 of the first finger connector 102 to the bottom portion (not shown) of the second finger connector 104, or vice-versa.

In some embodiments, in which the glove 10 is disposed atop the first and the second connector 102, 104, the interdigit guiding member 168 is attached onto the exterior of the glove 10. The interdigit guiding member 168 is positioned proximate to the openings 180, 182 of the glove 10 (which aligns with the openings 142, 144 of the respective first and second finger connectors 102, 104) such that the retention arms 170 may immediately releasably retain the string 116 outside of each opening 180, 182.

It is contemplated that, in an alternative embodiment, the interdigit guiding member 168 may not be positioned proximate to the opening 180 of the index finger 108 of the glove 10 and proximate to the opening 182 of the gloved middle finger. For example, the interdigit guiding member 168 may be disposed at a mid-portion (not shown) along the index finger 108 of the glove 10 and a mid-portion (not shown) along the middle finger 112 of the glove 10. Alternatively, in an alternative embodiment, the interdigit guiding member 168 may be positioned proximate to the opening 180 of the index finger 108 of the glove 10 to the mid-portion (not show) of the middle finger 112 of the glove 10, or vice-versa.

It is further contemplated, in an alternative embodiment, that the interdigit guiding member 168 may be discontinuous. In other words, there may be a plurality of interdigit guiding members 168 disposed in between the first finger connector 102 and the second finger connector 104 (either directly attached to the first and the second finger connectors 102, 104 or attached to the exterior of the glove 10). For example, there may be three interdigit guiding members 168, with each interdigit guiding member 168 having at least a portion of the interdigit guiding member 168 including the retention arms 170. A first interdigit guiding member being disposed on the first finger connector 102 (or the exterior surface of the glove 10), a second interdigit guiding member being disposed along the interdigit 176 of the glove 10, and a third interdigit guiding member being disposed on the second finger connector 104 (or on the exterior surface of the glove 10).

The interdigit guiding member 168 may be attached to the exterior surface of the first and the second finger connectors 102, 104, and the exterior surface of the glove 10 via biocompatible glue, knots, medical tape, or a combination thereof. In an alternative embodiment, the interdigit guiding member 168 may be integrally formed with the glove 10.

In other embodiments, the interdigit guiding member 150 may be provided without the glove 10. In such examples, the interdigit guiding member 150 will be connected to one or both of the first and second finger connectors 102, 104.

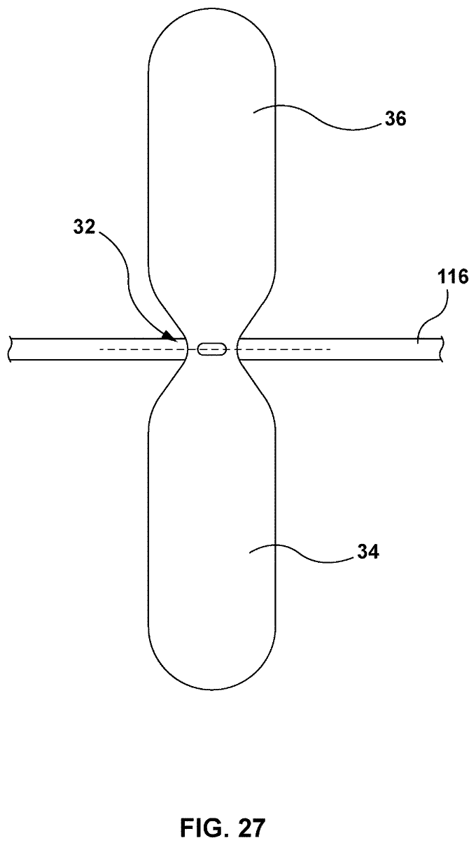

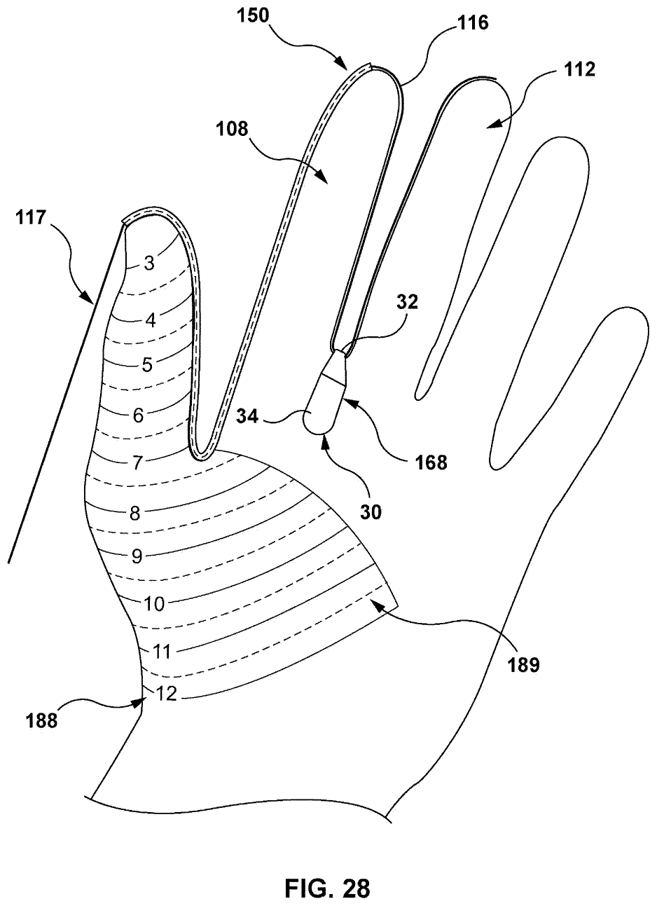

With reference to FIGS. 27 and 28, it is contemplated that the interdigit guiding member 150 may be configured as a breakable tab 30 disposed on the outer surface of the glove 10, in between the first and second connectors 102, 104. As depicted in FIG. 27, the breakable tab 30 includes a weakened portion 32 configured to tear or break, thereby releasing the string 116. In some embodiments, the weakened portion 32 includes at least one of: a frangible portion, a portion where there is less material, a portion with a smaller cross-sectional area, a perforated portion, or a combination thereof. The breakable tab 30 includes a first portion 34 which attaches to one portion of the outer surface of the glove 10 and a second portion 36 which attaches to another portion of the outer surface of the glove 10. The string 116 is positioned between the two portions 34, 36 of the breakable tab 30, held in place until the measurement device is ready to be used. For example, as seen in FIG. 28, the first portion 34 may attach to a portion of the glove 10 which is substantially on the top of the glove 10 while the second portion 36 may attach to a portion of the glove 10 which is substantially on the bottom of the glove 10 and the string 116 is disposed in between. During use, when the user is ready to begin measuring, the string 116 may be pulled from one end. As the string 116 tugs against the weakened portion 32 of the breakable tab 30, the weakened portion 32 breaks or tears, releasing the string 116.

### Locking Mechanism

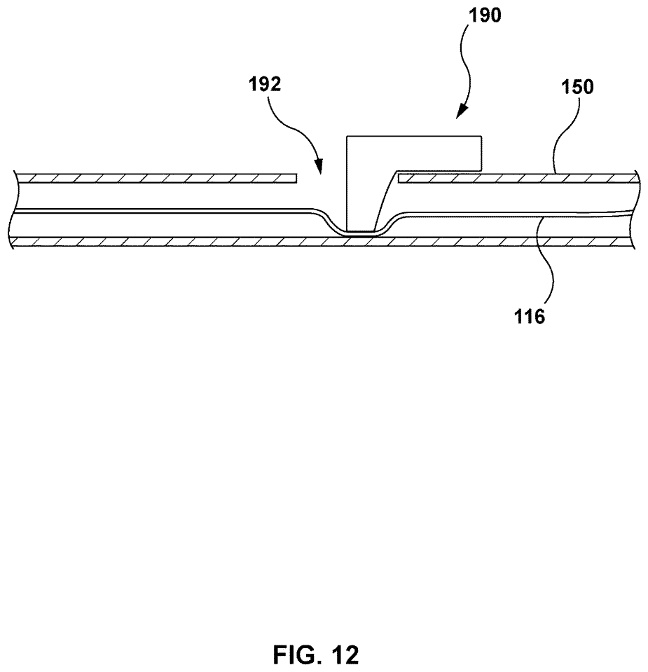

With reference to FIG. 12, the measurement device 100 may further include a locking mechanism 190 for releasably locking the string 116 in place, such as on the extension portion 117 thereof or on the interdigit guiding member 150.

In certain embodiments, the locking mechanism 190 is positioned along the index finger 108 and is configured to engage and disengage with a portion of the string 116. As depicted in FIG. 12, the locking mechanism 190 includes a sliding mechanism. The sliding mechanism aligns with an opening 192 of the tube 150 such that, when the sliding mechanism is actuated (e.g., sliding distally), a bottom surface 194 of the sliding mechanism engages with the string 116 (e.g., pinching the string) such that the string 116 is locked in place. The sliding mechanism can disengage the string 116 (e.g., sliding proximally) to release the string 116. In an alternative embodiment, the tube 150 may not include an opening 192 and, instead, the sliding mechanism pinches the string 116 indirectly via the tube 150 (not shown). It is contemplated that another locking mechanism may be used, including but not limited to, a button, a releasable twist-tie, a twisting pin, etc. It will be appreciated that the locking mechanism can be provided, even in the absence of the guiding member 150.

### Glove Measurement Markings

With reference to FIGS. 7, 9 and 10, the glove 10 of the measurement device 100 may further include measurement markings 188 disposed along one side of the exterior of the glove 10 for indicating a depth of insertion. The measurement markings 188 may be meter-like measuring marks disposed along the index finger 108, extending towards the wrist 132.

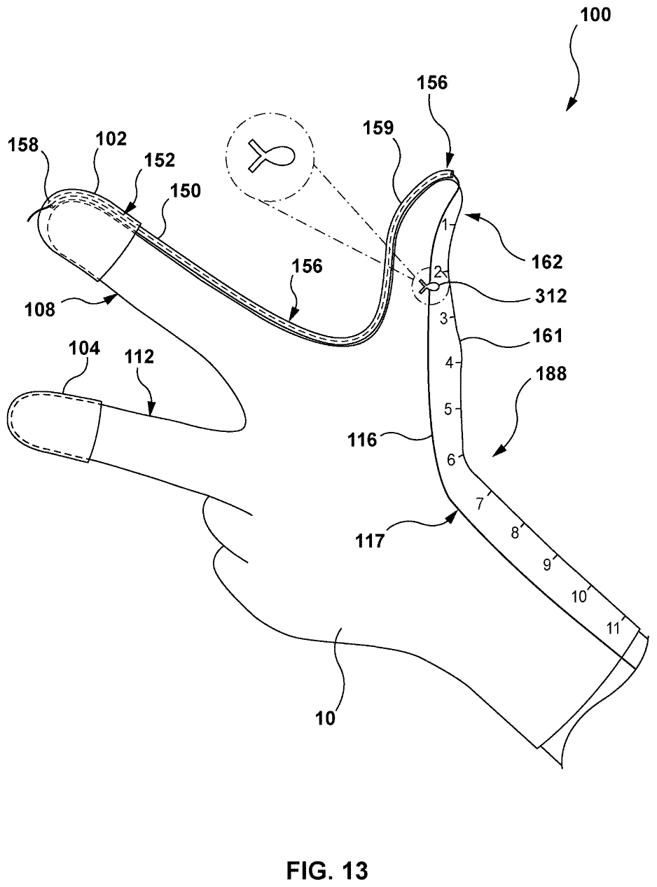

As depicted in FIG. 13, in an alternative embodiment, the glove 10 of the measurement device 100 includes the measurement markings 188 disposed along the thumb 162 of the glove 10. Specifically, the markings 188 may be disposed on an exterior surface of the thumb 162 of the glove 10 and along the top of the glove 10. It is contemplated that the measurement markings 188 may be disposed on both the index finger 108 of the glove 10 and the thumb 162 of the glove 10. Instead of, or in addition to, the above, the markings 188 may also be disposed on the palm of the glove 10. The markings 188 may be distance increments and/or color coded.

As depicted in FIG. 28, in alternative embodiments, the glove 10 may include measurement markings 188 and indication bands 189 extending at least partially across the top and at least partially across the bottom of the glove 10. For example, the indication bands and the measurement markings 188 may extend around a thumb portion of the glove 10. The indication bands 189 may be semi-circular. Specifically, the indication bands 189 provide a measurement scale and may alternate in color, corresponding to an increment of the measurement markings 188. In other embodiments, each increment of the measurement markings 188 may correspond to a specific color. The indication bands 189 may be formed in any manner such as by printing (e.g., pad printing or silkscreen printing). It is contemplated that, in further alternative embodiments, the measurement markings 188 may be omitted such that the glove 10 only includes the indication bands 189. It is further contemplated that the indication bands 189 may extend around an entire circumference of the glove 10, or across the entire top of the glove 10, or across the entire bottom of the glove 10. In alternative embodiments, the indication bands 189 and measurement markings 188 may be disposed along the wrist portion of the glove 10. It is appreciated that the measurement markings 188 may comprise any measure, such as but not limited to centimeters, half-centimeters, millimeters, inches, etc.).

It is appreciated that the first and the second connectors 102, 104, the string 116, the tube 150, and the interdigit guiding member 168 may be configured to be sterilized. In this instance, the components are releasably attached to the glove 10.

It is appreciated that the measurement device 100 may be manufactured in a variety of different sizes to accommodate the different hand sizes of various users. For example, a plurality of gloves 10 of different sizes may be provided. As the size of fingers and hands between users varies, the measurement device 100 may be calibrated to account for said differences.

### Sensors

In certain embodiments, the measurement device 100 may further include at least one sensor for collecting other data regarding the patient. In some embodiments, the sensor may detect at least one of a pH of the vaginal canal, a temperature of the vaginal canal, and/or a pressure within the vaginal canal.

In some embodiments, the sensor may be a colour changing sensor which changes colour when exposed to a certain pH level or temperature. The colour changing sensor may be positioned anywhere on the measurement device 100 which will be exposed to the vaginal canal. For example, in some embodiments, the colour changing sensor may be positioned on one of the fingers of the glove 10 (e.g., the index finger 108 and/or the middle finger 112 of the glove 10). It is appreciated that the colour changing sensor may be positioned on another portion of the measurement device 100.

In some embodiments, the sensor may be a pressure film. The pressure film may be disposed on a portion of the measurement device 100 which would contact the vaginal canal walls. For example, in some embodiments, the pressure film may be positioned on a lateral or side portion of the index finger 108 and/or the middle finger 112 of the glove 10, such that, as the user contacts the vaginal canal walls to obtain a measurement of distance between the vaginal canal walls, the user may also apply an outward pressure against said walls to determine a pressure exerted back onto the user. As pressure is applied onto the pressure film, the pressure film changes colour and/or colour intensity. When the user withdraws the measurement device 100 from the patient, they may compare the colour and/or intensity of the pressure film to a colour scale (which may be provided with the measurement device 100) to indicate a pressure level (e.g., low, medium, or high; or a scale of 1 to 10).

### Calibration Tool

In certain embodiments, the measurement device 100 may be pre-calibrated such that the indicator reflects the actual real-life separation distance. In alternative embodiments, there may be provided a calibration tool for calibrating the measurement device 100. The calibration mechanism will be described in further detail below.

The calibration tool may comprise a pre-measured object, such as a ruler, which can be compared against the separation distance as determined by the measurement device 100. The calibration tool can then be used to formulate and apply a calibration factor for translating the indicator measurement. Alternatively, the calibration factor can be used to adjust the indicator to ensure that the measurements obtained by the indicator accurately reflect the actual measurement of the separation distance.

In some embodiments, the calibration tool may include a rolling mechanism configured to be adjustable to the user's finger size. The user inserts their finger into the rolling mechanism and adjusts the roller shaft to the size of their finger which, in turn, may be used to determine the calibration factor for translating the indicator measurement.

In other embodiments, the calibration tool may include a finger size indicator. The user would compare their finger size with those displayed on the finger size indicator. Each finger size indicator is associated with the calibration factor for translating the indicator measurement.

In alternative embodiments, there may be an indication of the calibration factor associated with each measurement device 100 size available. For example, each glove size may be associated with a different calibration factor.

### Kits

In certain embodiments, there is provided a kit including: the measurement device 100 as described herein and the calibration tool for calibrating the measurement device 100. That is, each kit may include at least one of the calibration ruler, the rolling mechanism, the finger size indicator, and/or the indication of the calibration factor associated with the measurement device 100.

In certain embodiments, there is provided a kit including a plurality of the measurement devices 100 as described herein, at least some of the plurality of measurement devices 100 being different sizes, accommodating for different sizes of a user or of the patient.

In certain embodiments, the kit may further include a lubricant which may be applied to the string 116 and/or guiding member 150 to facilitate slidability of the string 116.

It is contemplated, that the measurement device 100 may include one or more of: a storage device for storing data obtained by the measurement device 100, a processor for processing the data obtained by the measurement device 100, or a network connector for sending and/or receiving data such as to an external database or an electronic health record of a patient. The storage device may be integrated with the measurement (“built-in”) or be separate thereto and connectable with. The network connector may be based on Wi-Fi, cellular, Bluetooth or any other type of connection. In certain embodiments, the indicator 114 may be a digital measuring tape for example, and include a processor for storing the separation distance measurement, or for sending the separation distance measurement such as to a server or a database holding patient medical records. The data obtained by the measurement device 100 may be processed to design a gynecological medical device such as a pessary. The obtained data may be used to train a machine learning algorithm.

It is contemplated, in an alternative embodiment, that the measurement device 100, the first and the second finger connectors 102, 104 may be configured to not include the openings 140, 142 at the center portion of the distal tips 144, 146. In another alternative embodiment, the first and the second finger connectors 102, 104 may include a channel disposed at the center portion of the distal tips 144, 146 of the respective connectors 102, 104 which is configured to receive the string 116. In this embodiment, the string 116 is extended through the channel of the first finger connector 102 and the second finger connector104, where it is affixed to the second finger connector.

In a further alternative embodiment, the first finger connector102 includes the slidable connection (e.g., the ring or the opening 142) with the string 116 and the second finger connector 104 includes the anchoring of the string 116 at the center portion 146 (that is, no ring or opening 144 is disposed on the second connector 104).

It is appreciated that any of the previously described embodiments of the first and the second finger connectors 102, 104 may be used in any arrangement. That is, for example, the first finger connector 102 may be configured with the opening 140, as previously described, and the second finger connector 104 may be configured with the ring. It is further appreciated that any other mechanism may be used for the connection between the first and the second finger connectors 102, 104 and the string 116 while providing the center-to-center configuration described previously.

In a further alternative embodiment, it is contemplated that the glove 10 may be omitted. In this instance, the components of the measurement device 100 may be removably attached to the user (e.g., using medical tape).

It is appreciated that the measurement device 100 described above is exemplary and alternative embodiments of the measurement device 100 may include any combination of one or more of the first finger connector 102, the second finger connector 104, the indicator 114, the string 116, the guiding member 150, the interdigit guiding member 168, the locking mechanism 190, and the glove 10. For example, it is contemplated that the first and second finger connectors 102, 104 may be omitted. In this example, the string 116 may pass through the guiding member 150 which is attached to the glove 10.

Although the measurement device 100 has been described with relation to the index finger 108, middle finger 112, and (if applicable) thumb 162, it is appreciated that the measurement device 100 may be configured to be used on other fingers. The fingers do not have to be adjacent to one another.

It is contemplated, in alternative embodiments, that the string 116 of the measurement device 100 may be replaced by a sensing system. For example, in some instances, the sensing system may include a first component associated with the first connector 102 and a second component associated with the second connector 104. The sensing system may further include a display, such as a screen or a mechanical indicator, which is communicatively connected to the first and second components. In some instances, the first and second components may be connected via wires. In some embodiments, the wires may be retractable. In other embodiments, the wires are fixed. In further embodiments, the first and second components may be connected wirelessly. The sensing system may further include a processor communicatively connected to the first and second components. In some examples, the first and second components may be positioned at a tip of the respective connectors 102, 104, such that the components align with the fingertips, and/or may be positioned at a lower portion of the respective connectors 102, 104, such that the components align with a midpoint of a respective finger. In some instances, the first and second components may include sensors which produce signals indicative of distance. For example, the sensors may be selected from at least one of: a Hall effect sensor, Bluetooth, RFID, and inertial measurement unit (IMU). The sensing system of the measurement device 100 may further communicate with a storage device for storing data obtained by the measurement device 100, a processor for processing the data obtained by the measurement device 100, or a network connector for sending and/or receiving data such as to an external database or an electronic health record of a patient, as previously described.

With reference to FIG. 20, a method 300 of using the measurement device 100 will now be described. The method 300 is described in relation to measuring distances between anatomical features in a vaginal canal of a patient. However, in other embodiments, the measurement device 100 can be used in relation to other anatomical parts. The method 300 will also be described in relation to the embodiment of the measurement device 100 in which the finger connectors comprise the first and second finger connectors 102, 104 having the string arranged therebetween in the center-to-center configuration.

Broadly, the method 300 comprises: inserting at least a portion of the measurement device 100 into a patient, the measurement device 100 comprising the first and second finger connectors 102, 104 respectively connected to first and second fingers of the user, the measurement device 100 having a string extending between the first and second connectors and having the extension portion extending beyond one of the first and second connectors (Step 302); positioning the first finger connector of the measurement device 100 at a first target location (Step 304); positioning the second finger connector of the measurement device 100 at a second target location (Step 306); determining the separation distance between the first finger connector and the second finger connector based on an extension length of the extension portion of the string, which extension length can be determined from outside of the patient (Step 308).

Step 302 of inserting the measurement device 100 into the patient includes inserting the measurement device 100 in a substantially closed configuration into the vaginal canal of the patient. That is, the first and second finger connectors are positioned close or adjacent to each other. Once inserted, the first and the second finger connectors 102, 104, and the string 116 extending therebetween, are inside the vaginal canal of the patient while at least the indicator 114 remains outside of the patient. During the process, the user references the indicator 114 disposed outside of the patient to obtain measurements, as will be described in further detail below. The indicator may be disposed on the extension portion 117 of the string, in the spool assembly, or be stored/communicated digitally.

At Step 304, the first finger connector 102 is positioned at the first target location. As the string 116 is disposed in the center-to-center configuration, positioning the first finger connector 102 includes positioning the center portion 140 of the distal tip 141 of the finger connector 102 at the first target location.

At Step 306, the second finger connector 104 is positioned at the second target location. Step 306 includes moving the second finger connector 104 away from the first finger connector 102 until the second target location is reached and positioning the second finger connector 104 the center portion 146 of the distal tip 147 of the second connector 104 at the second target location. Thus, a center-to-center measurement between the first and the second target location can be determined. It will be appreciated that the order in which the first and second finger connectors 102, 104 are positioned at their respective target locations is not limited. In some embodiments, the first and the second finger connectors of the measurement device 100 comprise a first and a second finger connector 102, 104.

As previously described, the center-to-center configuration of the measurement device 100 provides users with the ability to measure locations at a variety of angles with accuracy and reproducibility. In some embodiments the first target locations and the second target locations include: the ischial spines, the ischial spines and the coccyx, the ischial spines and the pubic symphysis, anterior and posterior fornices, the upper vagina, mid vagina and low vagina, mid-lower and medial vagina, width of the medial vagina, and width of the exocervix and exocervical canal. In another example, when measuring fetal descent, the target locations may include lateral ischial spine and pubic symphysis.

The method 300 includes step 308 of determining the extension length 137 using the indicator 114. Specifically, as the second finger connector 104 moves away from the first finger connector 102, and the string 116 slidably moves with respect to the first finger connector 102. As the separation distance between the first and second finger connectors 102, 104 increases, the extension length 137 of the extension portion 117 decreases. As mentioned earlier, the change in the extension length 137 of the extension portion 117 is proportional to the change in the separation distance 138, thereby permitting the separation distance 138 to be measured using the extension length 137 from the associated indicator 114.

In embodiments in which the indicator 114 comprises the spooled tape measure 126, the end 118 of the extension portion 117 of the string 116 is connected to the tape measure 126 of the spool assembly 115 indicator 114 (FIGS. 1 and 2). The change in the extension length 137 when the first and second finger connectors are separated is proportional to a change in the spooled length 138 of the tape measure. In other words, the modulation of the extension length 137 is proportional to the spooled length 138 which is proportional to the separation distance 136. As the tape measure 126 and the spool assembly 115 remain outside of the patient, the measure of the spool length 138 can be read off the tape measure 126.

In some embodiments, step 308 includes reading a measurement from the tape measure 126. In some embodiments, the spooled assembly may include digital readings of the spooled length which may be stored.

As described above, in certain embodiments, the extension length 137 is proportional to the spooled length 138 of the tape measure 126 which is proportional to the separation length 136. Thus, step 310 of determining the separation distance may include applying a calibration factor to the spooled length 138 to obtain the separation length 136. Broadly, the calibration factor is a predetermined factor for converting the measurement obtained by the indicator to the length of the extension portion and ultimately the separation distance. It will be appreciated that the extension length 137 and the separation length 136 may be directly proportional (i.e., 1 to 1). Obtaining the calibration factor will be described in further detail below.

In certain other embodiments in which there is no spool assembly, the extension portion 117 may include the visual indicators of measurement, such as numbers, color-coded markings etc., and the determining the separation distance comprises determining the extension length 137 from the visual indicators.

As the extension length 137 (or spooled length 138) which is indicative of the separation distance 138 remains outside the patient, the measurement device 100 can obtain multiple measurements without needing to be withdrawn from the patient. As such, the method 300 may include retracting the measuring tape 126 (e.g., by pressing a button on the measuring tape) which retracts the string 116. The user then repeats steps 302 to 310 to obtain measurements between other target locations (e.g., a third target location to a fourth target location). In other embodiments, the measuring tape may be biased to the spooled configuration so the measurement device 100 may be “re-set” between measurements simply by bringing the fingers closer together.

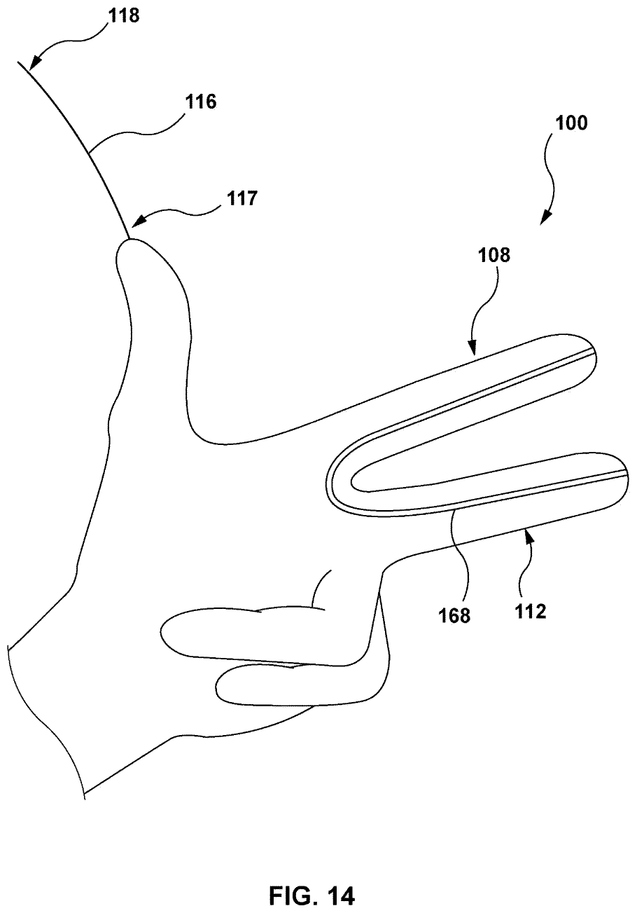

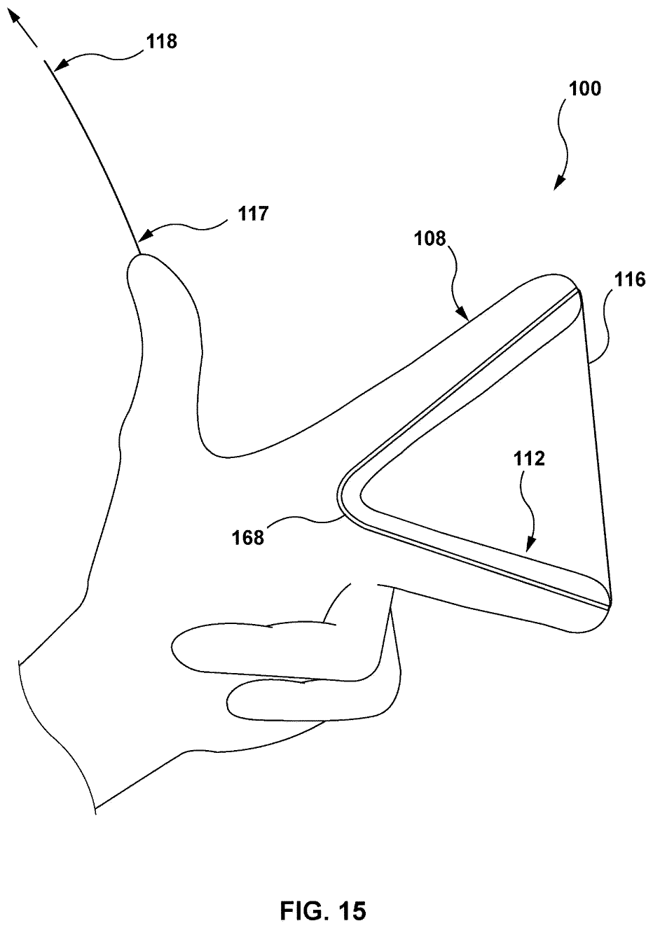

Referring to the embodiment of the measuring device 100 of FIGS. 14 and 15, in some embodiments, the method 300 may include releasing the string 116 from the retaining arms 170 of the interdigit guiding member 168 by pulling on the extension portion 117 of the string 116. The user may pull a region of the extension portion 117 of the string 116, such as the end 118, to pull the string 116 out of the retaining arms 170. The user continues to pull the end 118 of the string 116 until substantially all the string 116 is pulled through and the measurement device 100 is ready to be used. In certain embodiments, releasing the string 116 may be done before or after positioning the second finger connector 104 at the second target location.

In some embodiments, the method 300 may include reading a measurement from the measurement markings 188 along the glove 10. In certain embodiments, the measurement markings 188 are disposed along the index finger 108 of the glove 10 down the top side of the glove 10. In another embodiment, the measurement markings 188 may be disposed along the thumb 162 of the glove 10 (as seen in FIG. 13).

In certain embodiments, the method 300 may include injecting lubricant into the tube 150 of the measurement device. The measurement device 100 may include the injection hole for adding lubricant into the tube 150.

The method 300 may also include recording the measurements obtained by the measurement glove 100 in the patient's file. In some embodiments, the measurement device 100 may be configured to communicate with a patient database, such as an electronic health record, to automatically record and store the data collected.

As previously mentioned, the measurement of the extension length 137 (or spooled length 138) may require calibrating using the calibration factor. In some embodiments, calibrating the measurement device 100 includes measuring a calibration tool with the measurement device 100 (for example, a pre-measured object provided with the measurement device 100). Upon measuring the calibration tool, the user determines the difference between the actual measurement of the device (the pre-measured value) and the measurement obtained using the measurement device 100. This difference is the calibration factor which the user can apply to the extension length 137 (or spooled length 138) measurements to determine the separation distance 136.

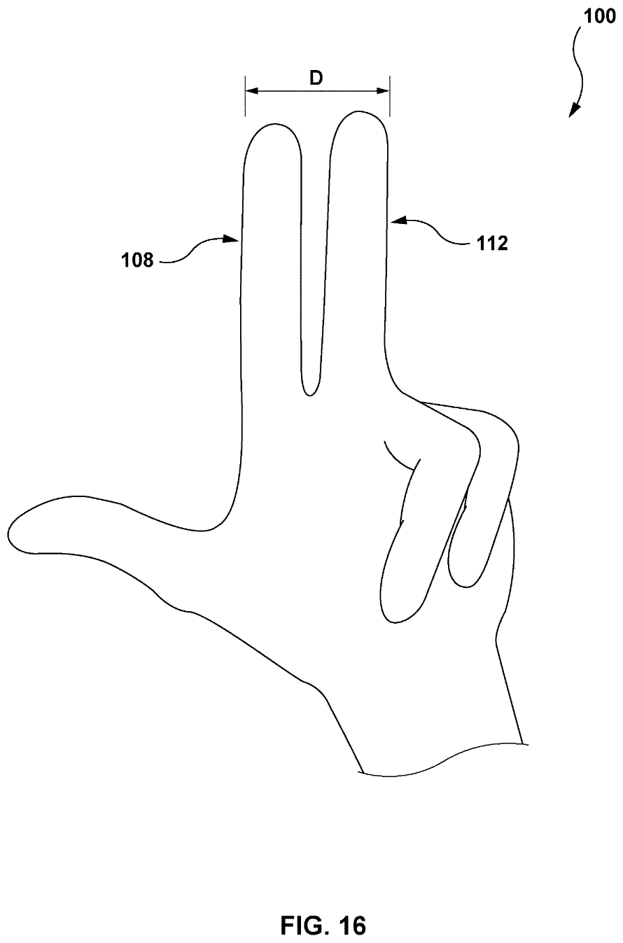

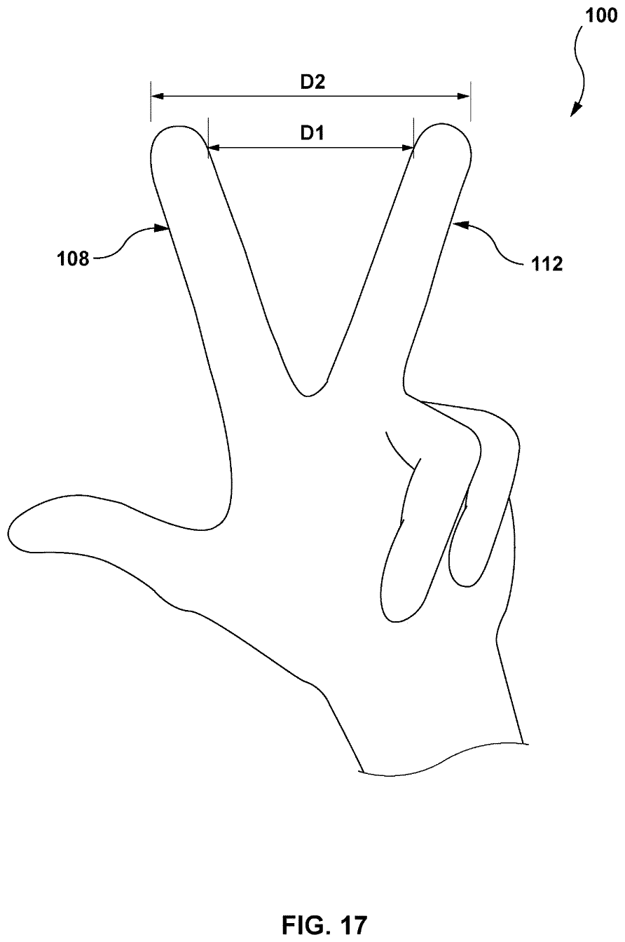

Referring to FIGS. 16 and 17, an alternative embodiment of calibrating the measurement device 100 is depicted. Calibrating the measurement device 100 may be done by determining the user's finger thickness and adding that value to the extension length 137 (or spooled length 138). As depicted in FIG. 16, determining finger thickness may be determined by taking the distance D between the outside of the index finger 108 of the user and the outside of the middle finger 112 of the user when the user's fingers are in the closed configuration. The user may then add each distance to their measurements to be used individually.

In addition to or in the alternative, as seen in FIG. 17, taking the distance D1 between the inside of the index finger 108 and the inside of the middle finger 112 and the distance D2 between the outside of the index finger 108 and the outside of the middle finger 112. The user can subtract the two distances and divide the subtracted value by two (i.e.,

$$\frac{D 2 - D 1}{2}$$

or D2/2) to determine the calibration factor to be added.

It is appreciated that calibrating the measurement device 100 may not be required if the measurement device 100 was pre-calibrated during manufacturing. For example, the calibration factor is provided by the manufacturer based on the glove 10 size the physician chooses. That is, each glove 10 size has the calibration factor correlated with it.

Although the method 300 is described for embodiments in which the measurement device 100 includes the tape measure 126, it is contemplated alternative steps may be taken for measurement devices 100 including a different indicator 114, for example, visual markings and/or tactile markings disposed on the extension portion 117 of the string 116.

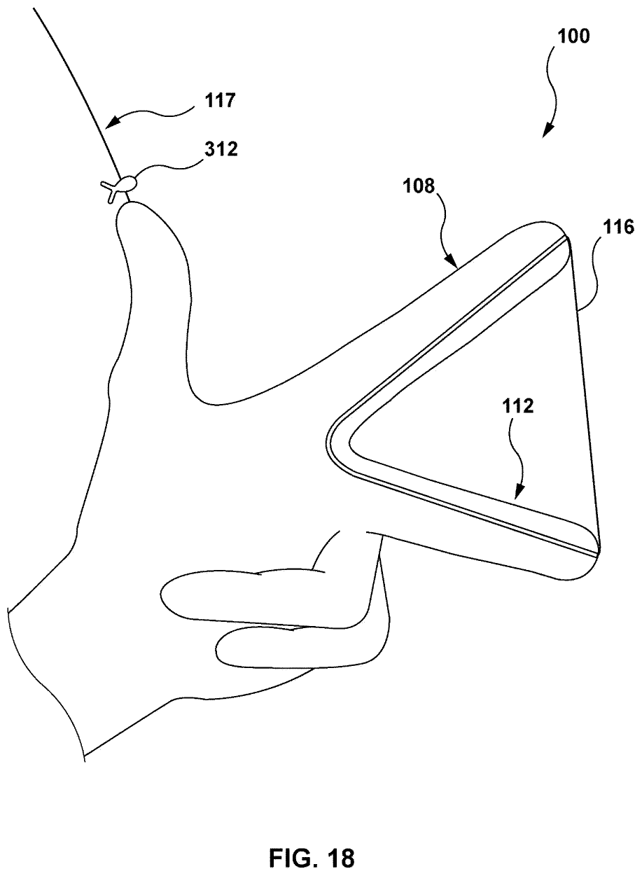

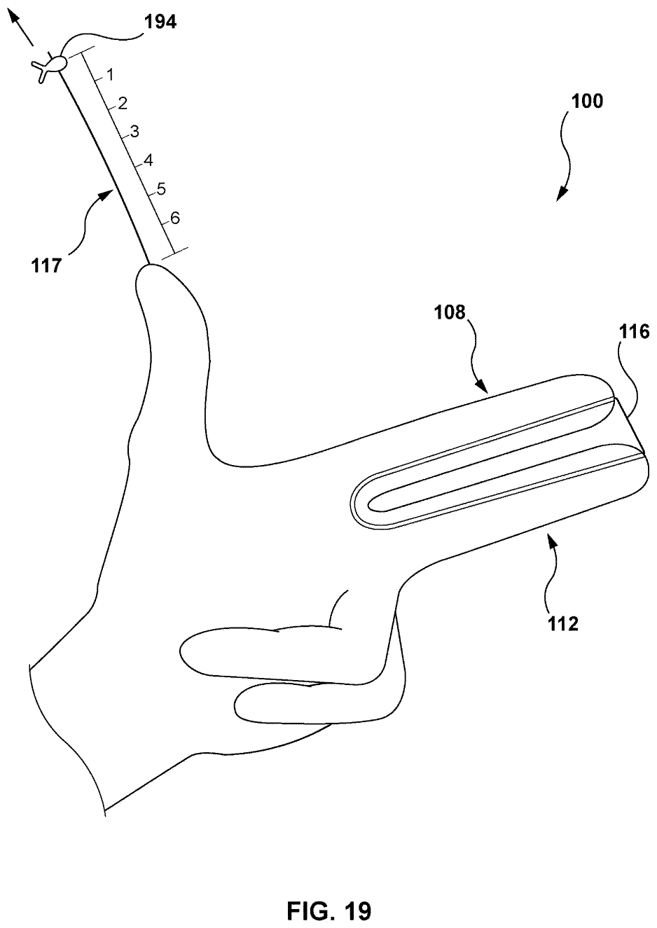

The alternative steps for the method 300 will now be described. With reference to FIGS. 18 and 19, the step 308 of determining the extension length 137 includes marking the string 116. In some embodiments, marking the string 116 includes applying a clipper 312 to the string 116. In an alternative embodiment, marking the string 116 may include pinching the string 116. In other embodiments, the clipper 312 may have any other configuration, or be omitted.

Step 310 of determining the separation distance 138 includes bringing the index finger 108 and the middle finger 112 together (i.e., in the substantially closed configuration) and measuring the extension portion 117 of the string 116 relative to the marking applied to the string 116. Specifically, measuring the extension portion 117 includes pulling the extension portion 117 and measuring the extension length 137 (denoted by the marking/clipper 312) using a measuring tool, such as a ruler, meter, pop-q, etc. Alternatively, the measuring tool may be incorporated into the measurement device 10. For example, having the meter-like markings 188 disposed along the length of the gloved thumb 162 (as depicted in FIG. 13).

Modifications and improvements to the above-described embodiments of the present invention may become apparent to those skilled in the art. The foregoing description is intended to be exemplary rather than limiting. The scope of the present invention is therefore intended to be limited solely by the appended claims.

What is claimed is:

1. A measurement device, comprising:
a first finger connector for connecting to a first finger;
a second finger connector for connecting to a second finger;
a string extending between the first finger connector and the second finger connector, and having an extension portion extending beyond at least one of the first and second finger connectors by an extension length;
wherein a separation distance of the first and second finger connectors is proportional to the extension length.

2. The measurement device of claim 1, wherein the string is anchored with respect to one of the first and second finger connectors and is slidingly moveable with respect to the other of the first and second finger connectors.

3. The measurement device of claim 1 or claim 2, further comprising an indicator for indicating a measure of the separation distance or the extension length.

4. The measurement device of claim 3, wherein the extension portion is connected to a spool assembly, the extension length being proportional to a spooled length, the indicator comprising one or more of: visual markings on the string or the spool assembly, tactile markings on the string or the spool assembly, and a digital/mechanical output of the spooled length.

5. The measurement device of claim 4, wherein the spool assembly comprises a tape measure device which is wearable on a wrist of the user.

6. The measurement device of claim 4 or claim 5, wherein the visual markings comprise one or more of: colours, indicia, letters or numbers.

7. The measurement device of any of claims 3 to 6, wherein the indicator comprises one or more of: visual markings on the string and tactile markings on the string.

8. The measurement device of claim 7, wherein the visual markings and/or the tactile markings are on the string.

9. The measurement device of any of claims 1 to 8, further comprising a hand covering, wherein the first finger connector and the second finger connector are integral with or disposed in relation to the hand covering.

10. The measurement device of claim 9, wherein the hand covering is disposed over the first and the second finger connectors, the hand covering comprising:
a first hand covering finger housing the first finger connector, the first hand covering finger having a first hand covering opening;
a second hand covering finger housing the second finger connector, the second hand covering finger having a second hand covering opening; and
the string extending through the first hand covering opening and the second hand covering opening.

11. The measurement device of claim 9, wherein the hand covering is disposed beneath the first and the second finger connectors, the hand covering comprising:
a first hand covering finger housed within the first finger connector; and
a second hand covering finger housed within the second finger connector.

12. The measurement device of claim 10, further comprising an additional hand covering, wherein the additional hand covering is disposed beneath the first and the second finger connectors.

13. The measurement device of any of claims 9 to 12, wherein the hand covering further comprises measurement markings disposed along an exterior surface of the hand covering, which markings may extend along one or more of the hand covering fingers and/or along a wrist portion of the hand covering.

14. The measurement device of any of claims 9 to 13, wherein the hand covering is reversible such that the hand covering can be worn on either a left or a right hand.

15. The measurement device of any of claims 10 to 14, further comprising a guiding member configured to receive the extension portion, the guiding member comprising a first end portion, a second end portion, and an intermediate portion therebetween.

16. The measurement device of claim 15, wherein the guiding member comprises a tube having a channel extending therethrough configured to receive the extension portion.

17. The measurement device of claim 15 or claim 16, wherein when the first finger connector is connected to the first finger of the user, and the second finger connector is connected to the second finger of the user:
the first end portion is disposed along the first finger; and
the second end portion is disposed along a wrist portion of the.

18. The measurement device of claim 17, wherein the intermediate portion is disposed along a third finger of the hand covering.

19. The measurement device of claim 15 or claim 16, wherein when the first finger connector is connected to the first finger, and the second finger connector is connected to the second finger:
the first end portion is disposed along a portion of the first finger of the hand covering;
the second end portion is disposed along a portion of a third finger of the hand covering.

20. The measurement device of claim 18 or 19, wherein the third finger of the hand covering is configured to receive a thumb when the measurement device is in use.

21. The measurement device of any of claims 15 to 20, wherein the first end portion arcs over a distal tip of the first fingertip when the measurement device is in use.

22. The measurement device of any of claims 15 to 21, wherein the first end portion comprises an opening, and the string extends through the opening.

23. The measurement device of any of claims 15 to 22, wherein the guiding member and/or the string is lubricated.

24. The measurement device of any of claims 15 to 23, wherein the guiding member comprises an injection port for injecting lubricant.

25. The measurement device of any of claims 1 to 24, wherein:
the first finger connector comprises:
a first distal tip having a first center portion;
the second finger connector comprises:
a second distal tip having a second center portion; and
the string extends from the first center portion of the first finger connector to the second center portion of the second finger connector.

26. The measurement device of any of claims 1 to 25, wherein an end region of the string is anchored in relation to the second finger connector.

27. The measurement device of any of claims 1 to 26, wherein the first finger connector comprises a first finger cap configured to accommodate a first fingertip of the first finger, and the second finger connector comprises a second finger cap configured to accommodate a second fingertip of the second finger.

28. The measurement device of claim 27, wherein the first finger cap comprises a first finger cap opening, and the string is disposed beneath the first finger cap and extends through the first finger cap opening.

29. The measurement device of claim 27 or claim 28, wherein the second finger cap comprises a second finger cap opening, and the string extends from the first finger cap through the second finger cap opening.

30. The measurement device of claim 29, wherein the string is affixed on or beneath the second finger cap.

31. The measurement device of any of claims 1 to 30, further comprising an interdigit guiding member disposed between the first finger connector and the second finger connector, the interdigit guiding member comprising at least one pair of retaining arms for releasably retaining the portion of the string extending between the first and second finger connectors such that the string extends along the interdigit guiding member.

32. The measurement device of claim 31, wherein the pair of retaining arms extend an entire length of the interdigit guiding member.

33. The measurement device of claim 31, wherein the interdigit guiding member comprises a plurality of pairs of retaining arms spaced apart along the interdigit guiding member.

34. The measurement device of any of claims 1 to 33, wherein the measurement device is configured to store the separation distance in a patient database or to cause the storing of the separation distance.

35. The measurement device of any of claims 1 to 34, further comprising a locking mechanism for securing a position of the extension portion of the string relative to one of the first and second finger connectors.

36. The measurement device of claim 35, wherein the locking mechanism is disposed along the extension portion of the string.

37. The measurement device of claim 35 or claim 36, wherein the locking mechanism presses down on a segment of the extension portion of the string upon actuation of the locking mechanism.

38. The measurement device of any of claims 1 to 37, wherein the first finger is an index finger and the second finger is a middle finger.

39. A kit comprising:
the measurement device of any one of claims 1 to 38; and
a calibration tool for calibrating the measurement device.

40. A kit comprising:
a plurality of the measurement device of any one of claims 1 to 38, each of the plurality of measurement device being different sizes, accommodating for different hand sizes; and
each of the plurality of measurement devices being pre-calibrated.

41. A method of using a measurement device comprising:
inserting at least a portion of a measurement device into a patient, the measurement device comprising first and second finger connectors, a string extending between the first and second finger connectors and having an extension portion extending beyond one of the first and second connectors, and an indicator associated with the extension portion;
positioning the first finger connector at a first target location;
positioning the second finger connector at a second target location so that the first and second connectors are separated by a separation distance;
determining the separation distance between the first finger connector and the second finger connector based on an extension length of the extension portion, which extension length can be determined from outside of the patient.

42. The method of claim 41, wherein positioning the first finger connector of the measuring device at the first target location comprises positioning a first center portion of a first distal tip of the first finger connector at the first target location.

43. The method of claim 41 or claim 42, wherein positioning the second finger connector of the measuring device at the second target location comprises positioning a second center portion of a second distal tip of the second finger connector at the second target location.

44. The method of any of claims 41 to 43, wherein the indicator comprises a spooled tape measure connected to an end of the extension portion of the string, and wherein determining the extension length comprises reading a spooled length from the tape measure, the spooled length being proportional to the separation distance.

45. The method of any of claims 41 to 43, wherein the indicator comprises at least one of visual markings and tactile markings disposed on the extension portion; and wherein determining the extension length comprises reading at least one of the visual markings and the tactile markings from the extension portion.

46. The method of any of claims 41 to 45, further comprising calibrating the measurement device using a calibration tool.

47. The method of any of claims 41 to 46, further comprising releasing the string from retention arms of the measuring device.

48. The method of claim 47, wherein releasing the string from the retention arms comprises pulling a first end of the string.

49. The method of any one of claims 41 to 48, further comprising:
moving the second finger connector towards a third target location, such the extension portion of the string of the measurement device extends a second extension length;
determining the second extension length; and
determining a second separation distance between the first finger connector and the second finger connector based on the second extension length.
